(19) United States
(12) Patent Application Publication
(10) Pub. No.: US 2023/0326571 A1
(43) Pub. Date: Oct. 12, 2023

(54) COMPUTATIONAL MODEL, METHOD, SYSTEM AND EXERCISE FRAMEWORK FOR ELEVATING CONSCIOUSNESS, INNER SYNCHRONIZATION AND GENERAL WELLNESS

(71) Applicant: AVRAHAM RAM GUISSIN, BEIT YANAI (IL)

(72) Inventor: AVRAHAM RAM GUISSIN, BEIT YANAI (IL)

(21) Appl. No.: 18/130,925

(22) Filed: Apr. 5, 2023

Related U.S. Application Data
(60) Provisional application No. 63/327,807, filed on Apr. 6, 2022.

(51) Int. Cl. G16H 20/30; G16H 40/67

(52) U.S. Cl. CPC G16H 20/30; G16H 40/67

(57) ABSTRACT

The awareness of inner change is an important mental function of the human condition that is affected by personality traits and personal circumstance. This invention relates generally to a physical and mental exercise and computational framework for progressively elevating the practitioner's levels of alertness, attentiveness and awareness of his/her cognitive (thoughts and intuitions) and perceptive (sensations and feelings) mental faculties as they function and respond to such inner change. Such elevated levels of consciousness enable the user to quantify experiential intensities with greater subjective efficacy, which are then entered into a computerized system that algorithmically computes and generates experiential maps of the user's impressions and transitions in selected underlying processes and themes. This in turn provides an introspective behavioral self-help guidance tool (e.g. mobile and desktop software application) for better comprehending one's evolving human condition, while retaining inner balance, resilience and general wellness.

### FIELD

The awareness of inner change is an important mental function of the human condition that is affected by personality traits and personal circumstance. This invention relates generally to a physical and mental exercise and computational framework for progressively elevating the practitioner's levels of alertness, attentiveness and awareness of his/her cognitive (thoughts and intuitions) and perceptive (sensations and feelings) mental faculties as they function and respond to such inner change. Such elevated levels of consciousness enable the user to quantify experiential intensities with greater subjective efficacy, which are then entered into a computerized system that algorithmically computes and generates experiential maps of the user's impressions and transitions in selected underlying processes and themes. This in turn provides an introspective behavioral self-help guidance tool (e.g. mobile and desktop software application) for better comprehending one's evolving human condition, while retaining inner balance, resilience and general wellness.

### BACKGROUND

The awareness of inner change is an important mental function of the human condition that is affected by personality traits and personal circumstance. The heightening of such inner awareness by combining exercise and technology provides introspective behavioral self-help guidance tools in the form of mobile and desktop software applications (APPs) for improved 1 comprehension of one's evolving human condition, while retaining inner balance, strengthening personal resilience and sustaining general wellness.

This invention provides a novel physical and mental exercise and computational framework for progressively elevating the practitioner's levels of alertness, attentiveness and awareness of his/her cognitive (thoughts and intuitions) and perceptive (sensations and feelings) mental faculties as they function and respond to such inner change. The methodology combines Yogic philosophy, Jungian psychology and Gestalt principles, in activating, synchronizing and coordinating body, breath and mental (BBM) faculties, that together create a sense of energized BBM coherence and resonance.

Such Synchronizing Inwards (SyncIn) of the BBM faculties is a key element of this invention, as these faculties are differently characterized in terms of their respective energies, rhythms, patterns and combined affects. A preferred embodiment of this invention is the use of temporal rhythmic patterns and respective inner speech orations in combined and synchronized activation of the BBM faculties. Specific sequenced inner speech orations are used in mindful Movement (MV) exercises that focus on activation and synchrony of the body (lower and upper limbs) and breath faculties based on the high-rate mental modality of Alertness. In turn, other and more contextual inner speech orations are utilized in mindful Meditation (MD) exercises that emphasize the cognitive and perceptive mental faculties of volitional thoughts and feelings, and spontaneous sensations and intuitions, based on the slower-rate mental modalities of Attentiveness and Awareness. Such contextual focus refers and relies on the preferred embodiment of this invention of (i) generic Themes and (ii) a generic experiential Construct/Process that comprises subsequent traceable States, that both constitute the context upon which the MD exercises meditate upon in synchronizing and quantifying the subjective intensity of rational and emotional human experiences.

Such elevated conscious levels of Alertness, Attentiveness and Awareness enable a user to quantify experiential intensities with greater subjective efficacy, and preferably can be subsequently used by a computerized algorithm-based system that computes and generates experiential maps of the user's impressions and transitions in selected underlying Processes and Themes. This in turn provides an introspective behavioral self-help guidance tool (e.g. mobile and desktop software APP) for better comprehending one's evolving human condition, while retaining inner balance, resilience and general wellness.

### SUMMARY

The following presents a simplified summary of the invention in providing a basic understanding of some aspects and implementations of the invention.

This invention takes a practical approach, whereby preferably a SyncIn APP that is driven by Machine Learning (ML) algorithms, is utilized in activating and synchronizing the BBM faculties of a user thru MV and MD exercises, thereby exercising the Executive Function of Self (EFS), that is otherwise and typically driven by motivations, and aimed at designated goals and expressed as behavior.

The EFS refers to the internal capacity to choose and to direct one's own behavior. The Self is a person's idea of who he/she is, and which is impacted by sensations, intuitions, thoughts, feelings, actions, and many other factors. The Self is assumed to have two functions: (a) the EFS function which helps regulate behavior, and (b) the organizational function which helps uncover patterns in the world. The EFS is well established by brain, cognitive and behavioral research, and is conceived as the hierarchical mental function that controls, monitors and manages the BBM faculties thru divided alertness, attentiveness and awareness. Exemplary acquired skills of the EFS include the abilities (a) to initiate tasks, (b) to self-monitor (i.e. evaluate and appraise) one's behavior, (c) to regulate/direct/maintain one's alertness, attention and awareness, (d) to hold, manage and access working memory information in performing tasks, (e) to plan and prioritize steps and processes in reaching one's goals, (f) to control and manage one's emotions in directing behavior, (g) to keep track of information, tasks and multi-tasking, (h) to think and contemplate before acting.

Therefore, for a SyncIn APP to be useful in exercising the EFS, its framework should include relevant (i) body functions and attributes (e.g. action, movement, organs of action, organs of perception), (ii) breath functions and attributes (e.g. tempo, intensity, flow, rhythm, organs and pathways of respiration), and (iii) mental faculties and attributes (e.g. sensating, intuiting, thinking, feeling and inner speech).

This invention describes the methods, technical means and process mechanisms that facilitate a diverse range of BBM exercise procedures (i.e. activities designed to develop executive function skills) and practice procedures (i.e. repetition of such activities) in improving and refining such EFS skills. In accordance with this invention, a preferred embodiment of such exercise and practice framework is guided and controlled by computerized software that is preferably configured, for example, as a mobile or PC/Tablet APP. The APP technology utilizes, for example, camera, microphone, tactile, movement, breath flow sensors, sound/voice sensors and algorithms in collecting, parametrizing and interpreting user exercise and practice measurable information and manually entered text and verbal speech information for feedback, recording, machine learning of user characteristics, practice scoring and guidance for improvement in terms of wellness criteria.

Another object of the current invention provides improved employee identification with the workplace and respective product lifecycles on which they are working. This also results in improved employee screening and placement at the workplace, hence increasing employees' mental wellbeing, work motivation, identification with products and projects in which they are involved, and general employment satisfaction and contribution.

These and other advantages and features of the present invention will become apparent from the following detailed descriptions and the attached drawings.

### BRIEF DESCRIPTION OF THE DRAWINGS

In order to understand the invention and to see how it may be carried out in practice, embodiments will now be described, by way of non-limiting example only, with reference to the accompanying drawings, in which:

FIG. 1 illustrates a high-level functional flow of the current invention, whereby an APP exercises and activates the EFS, which in turn communicates and activates the BBM faculties, in accordance with this invention;

FIG. 2 illustrates the underlying motivation and outcome of the exercise framework, in accordance with this invention;

FIG. 3 illustrates the concentric five-sheath representation of the human model according to ancient Yogic philosophy, and the extension of the model in accordance with this invention;

FIG. 4 depicts a full, eight-State Construct embodiment in accordance with this invention;

FIG. 5 illustrates a preferred representation of the EFS behavioral functionalities, in accordance with this invention;

FIG. 6 illustrates a preferred representation of the framework of the EFS, in accordance with this invention;

FIG. 7 illustrates a combined variety of Subprocesses and Transitions of Mind/Ego Faculties, and synchronized Breath, Organs of Action and Organs of Perception, all of the Construct and in accordance with this invention;

FIG. 8 Unit 810 illustrates the four mental functions (thinking, feeling, sensating and intuiting), and their categorizations and transitions in terms of (i) cognition and perception, and (ii) experience and presence, in accordance with this invention;

FIG. 9 illustrates the SyncIn APP as it interacts with the EFS model, in accordance with this invention;

FIG. 10 illustrates the functional layout and conceptual design of the SyncIn exercise APP, comprising three main processes namely Exercise Selector and Generator process, Conscious User Practice and Processing process, and Analysis and Scoring process.

FIG. 11 depicts the use of imaging and image processing technology in analyzing finger tap exercise accuracy and performance, in accordance with this invention;

FIG. 12 depicts the use of imaging and image processing technology in analyzing hand taps synchronized with sound beats, in accordance with this invention;

FIG. 13 illustrates the use of acoustic air flow sensing and processing of breath cycles in analyzing exercise quality and performance, in accordance with this invention;

FIG. 14 illustrates the process of training the user's speech modalities, in accordance with this invention;

FIG. 15 illustrates an exemplary SyncIn exercise as it compares to a Yoga Asana, in accordance with this invention;

FIG. 16 presents a block diagram that describes an input procedure of mental attributes of an experiential Process or an experiential Instance, including all the elements of the Construct, thereby updating the data structure of said Process or Instance, in accordance with this invention;

FIG. 17 presents a flow diagram that describes experiential Instance information accumulated over time, in accordance with this invention;

FIG. 18 illustrates an exemplary circular concentric representation of a basic SyncIn mindful MV exercise, in accordance with this invention;

FIG. 19 illustrates an exemplary circular concentric representation of SyncIn State-based mindful MD exercise, in accordance with this invention;

FIG. 20 illustrates an exemplary set of inner speech sequences, whereby the first five exercises aim to train the novice, and the last two sequences relate to State #1 and State #5, in accordance with this invention;

FIG. 21 illustrate various one-dimensional to n-dimensional graphical representations of computed State, Function and attribute intensity statistics, in accordance with this invention.

FIG. 22 illustrates exemplary user entries of intensities using MD inner speech exercises that activate queries using the four mental Functions in pairs: (a) dual Thoughts and Feelings inner speech sequences and (b) dual Sensations and Intuitions inner speech sequences, and whereby six generalized themes that are meditated upon are of general interest to all, and which reflect our personality traits, in accordance with this invention.

FIG. 23 illustrates exemplary user entries of intensities using MD inner speech exercises that activate experiential queries using the eight basic emotions in dual pairs (Anger-Fear, Joy-Sadness, Anticipation-Surprise and Trust-Disgust, for each of the eight States of the Experiential Process, in accordance with this invention.

FIG. 24 illustrates exemplary user entries of intensities using MD inner speech exercises that activate queries using the four mental Functions in pairs: (a) dual Thoughts and Feelings inner speech sequences and (b) dual Sensations and Intuitions inner speech sequences, and whereby the eight generalized queries that are meditated upon are the eight States of the Experiential Process and the theme is related to a relationship, in accordance with this invention.

FIG. 25 illustrates exemplary user entries of intensities using MD inner speech exercises that activate queries using the four mental Functions in pairs: (a) dual Thoughts and Feelings inner speech sequences and (b) dual Sensations and Intuitions inner speech sequences, and whereby the eight generalized queries that are meditated upon are the eight States of the Experiential Process and the theme is related to the business aspects of a project, in accordance with this invention.

FIG. 26 illustrates exemplary user entries of intensities using MD inner speech exercises that activate queries using the four mental Functions in pairs: (a) dual Thoughts and Feelings inner speech sequences and (b) dual Sensations and Intuitions inner speech sequences, and whereby the eight generalized queries that are meditated upon are the eight States of the Experiential Process and the theme is related to the theoretical aspects of a project, in accordance with this invention.

FIG. 27 illustrates two block diagrams of computational algorithms comprising (i) a Process and instance referencing phase and classification phase based on manually-entered experiential attributes in accordance with this invention, and (ii) a classical machine learning framework known in the art that combines a training phase and a prediction phase, in accordance with this invention;

FIG. 28 illustrates (i) a structured data table of attribute entries of a modeled Process, for each of the eight States and accumulated in multiple instances, thereby facilitating computing numerical statistics for each of the said attributes in each State, and (ii) a structured data table of said computed statistics of multiple differently-labeled Processes that is useful in classifying and associating different input instances to one of said labeled Processes, in accordance with this invention;

FIG. 29 presents an exemplary structured data table of the attribute statistics of the conscious Mind and Ego mental motivators, compiled in time periods that reflect progressive designated stages of an intimate relationship Process model, for each of the eight States, thereby facilitating classification and various assessments of said relationship Process, in accordance with this invention;

FIG. 30 illustrates a mobile cellular device application that facilitates the use of the experiential Construct in everyday life by an end user and by a professional therapist, and the app's connectivity to a central server and other devices, in accordance with this invention;

FIG. 31 illustrates an exemplary SyncIn APP display and user interfaces, in accordance with this invention;

FIG. 32 illustrates the mobile device application as an experiential data entry assistant by using the graphical Construct, in accordance with this invention;

FIG. 33 illustrates the utility of the current invention, that provides improved employee identification with the workplace and respective product lifecycles on which they are working, in accordance with this invention.

FIGS. 34-38 list for reference a variety of SyncIn practice sessions.

FIG. 39 is a wheel-like map depicting Plutchik's feelings that are generally composed of two emotions.

### DETAILED DESCRIPTION

This invention takes a practical approach, whereby preferably a SyncIn APP that is driven by ML algorithms, is utilized in activating and synchronizing the BBM faculties of a user thru MV and MD exercises, thereby exercising the EFS, that is otherwise and typically driven by motivations, and aimed at designated goals and expressed as behavior.

The EFS refers to the internal capacity to choose and to direct one's own behavior. The Self is a person's idea of who he/she is, and which is impacted by sensations, intuitions, thoughts, feelings, actions, and many other factors. The Self is assumed to have two functions: (a) the EFS function which helps regulate behavior, and (b) the organizational function which helps uncover patterns in the world.

The EFS is well established by brain, cognitive and behavioral research, and is conceived as the hierarchical mental function that controls, monitors and manages the BBM faculties thru divided alertness, attentiveness and awareness. Exemplary acquired skills of the EFS include the abilities (a) to initiate tasks, (b) to self-monitor (i.e. evaluate and appraise) one's behavior, (c) to regulate/direct/maintain one's alertness, attention and awareness, (d) to hold, manage and access working memory information in performing tasks, € to plan and prioritize steps and processes in reaching one's goals, (f) to control and manage one's emotions in directing behavior, (g) to keep track of information, tasks and multi-tasking, (h) to think and contemplate before acting.

Therefore, for a SyncIn APP to be useful in activating and exercising the EFS, its framework should include relevant (i) body faculties, functions and attributes (e.g. action, movement, organs of action, organs of perception, rhythm), (ii) breath faculties, functions and attributes (e.g. tempo, intensity, flow, rhythm, organs and pathways of respiration), and (iii) mental faculties, functions and attributes (e.g. sensating, intuiting, thinking, feeling, rhythm and inner speech).

This invention describes the methods, technical means and process mechanisms that facilitate a diverse range of BBM exercise procedures (i.e. activities designed to develop EFS skills) and practice procedures (i.e. repetition of such activities) in improving and refining such EFS skills. In accordance with this invention, a preferred embodiment of such exercise and practice framework is guided and controlled by computerized software that is preferably configured, for example, as a mobile or PC/Tablet APP. The APP technology utilizes, for example, camera, microphone, tactile, movement, breath flow sensors, sound/voice sensors and algorithms in collecting, parametrizing and interpreting user exercise and practice measurable information and manually entered text and verbal speech information for feedback, recording, machine learning of user characteristics, exercise scoring and guidance for improvement in terms of defined wellness criteria.

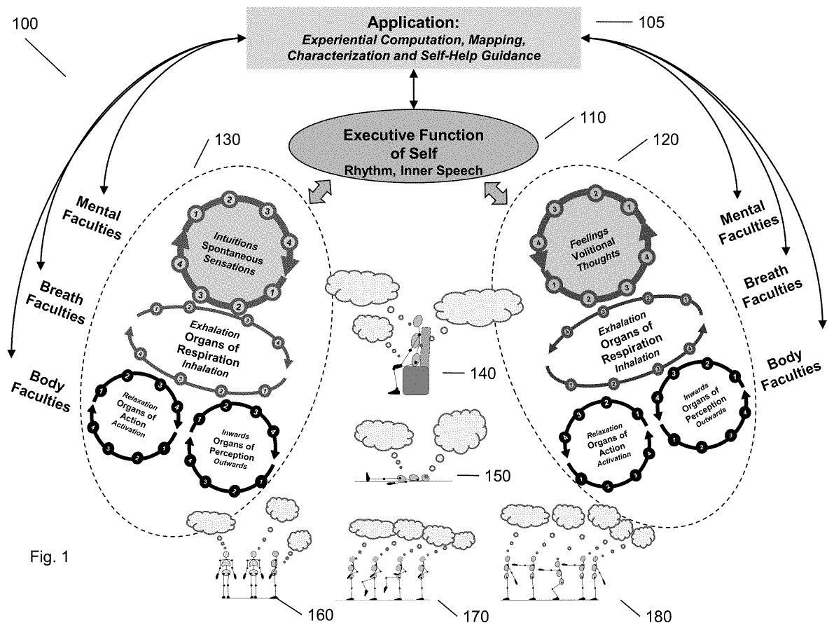

FIG. 1 illustrates a high-level functional flow of the current invention, whereby an APP Unit 105 that provides experiential computation, mapping, characterization, and self-help guidance to a user, by rhythmically and synchronously activating and exercising the user's BBM faculties such that EFS functioning is facilitated. In a preferred embodiment, the BBM functions are separated into a Volitional organic functioning Unit 120, and a Spontaneous organic functioning Unit 130. Each such Unit comprises body faculty functions (organs of action and organs of perception), breath faculty functions (organs of respiration), and mental faculty functions (Volitional Thought and Feeling functions, and Spontaneous Sensation and Intuition functions). Units 120, 130 are each cyclical and repetitive, and are governed by exemplary four beats per half cycle. For example, Unit 120 functioning synchronizes body Organs of Action (OOA) and Organs of Perception (OOP) with breath Organs of Respiration (OOR) and with Volitional Thoughts (VT) and Volitional Feelings (VF). In this exemplary preferred embodiment, temporal rhythm beats are synchronized with inhalation and exhalation breath cycles, with body limb activation and relaxation movements, with body outwards and inwards perception, and with respective VT and VF functions. Unit 140 presents a seated exercise position, Unit 150 presents a supine exercise position, and Units 160, 170 and 180 present exemplary standing and walking exercise positions.

The APP is therefore designed to both activate, exercise monitor rhythmic and synchronized faculty functioning, in assisting and training the EFS such that it can alternatively function independently of the APP upon need in every day life functions.

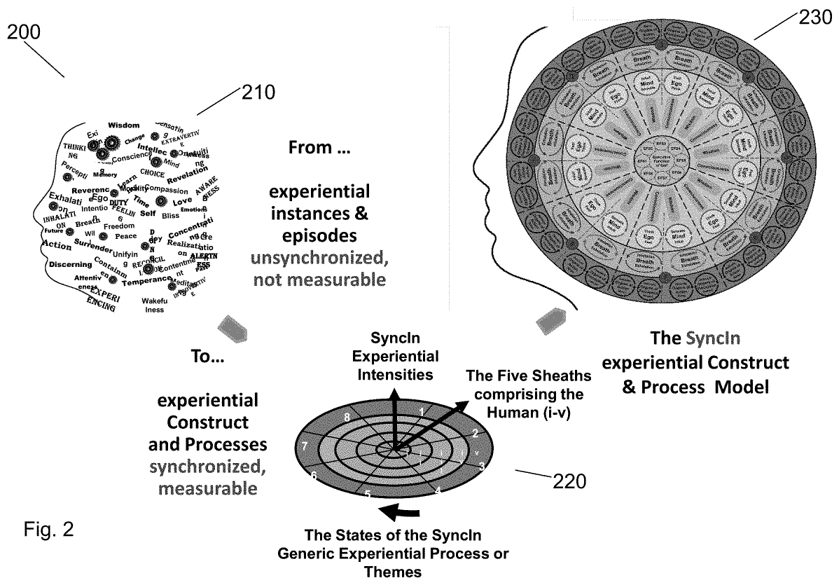

FIG. 2 illustrates the underlying motivation and desired outcome of the exercise framework. Unit 210 illustrates the human “monkey mind” condition where one is called to rapidly skip, for example, between thoughts and feelings, and between sensations and intuitions. Such experiential processes comprise instances and episodes, which are often unsynchronized and not measurable. Unit 220 illustrates a structured experiential Construct and Process that facilitates such functional BBM synchronization and experiential measurability and quantification. Unit 230 illustrates a preferred embodiment of this invention comprising structured experiential Construct and Process that facilitates such functional BBM synchronization and experiential measurability and quantification.

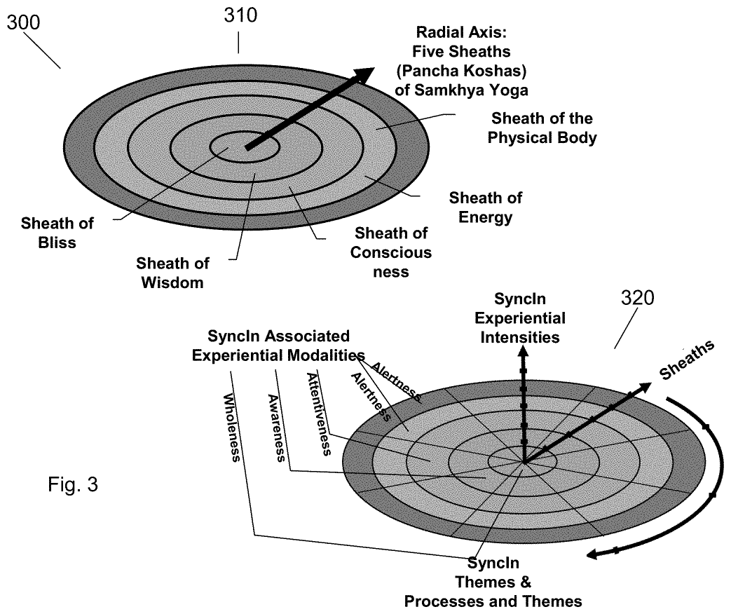

FIG. 3 illustrates the concentric five-sheath representation of the human model according to ancient Yogic philosophy, and the extension of the model in accordance with this invention. The human Construct as provided in this invention and illustrated in Unit 320 of FIG. 3, first builds on the well-known (prior art) concentric, five-sheath ancient Samkhya Yoga model as illustrated in Unit 310 that comprises from the outermost to the inner most sheath—the sheath of the physical body, the sheath of energy, the sheath of consciousness, the sheath of wisdom and the innermost sheath of bliss. These five sheaths (‘pancha koshas’ in Sanskrit) are believed to holistically underlie all aspects of the human paradigm.

The invented (new art) Construct of Unit 320 expands the two dimensional known circular concentric five-sheath model into a more comprehensive three-dimensional representation having three axis, namely (i) the radial axis of the five sheaths, (ii) the angular axis of Processes and Themes, and (iii) the vertical axis of experiential intensities. In accordance with this invention, the five sheaths are associated with experiential modalities as follows: Body and energy sheaths are associated with the modality of Alertness, the sheath of Consciousness is associated with the modality of Attentiveness, the sheath of Wisdom is associated with the modality of Awareness, and the sheath of Bliss is associated with the modality of Wholeness. These modalities will be further elaborated in this disclosure. In the Alertness Level we use the Discerning Modality in making distinctions between experiential instances and occurrences, and synchronizing basic ongoing Subprocesses (e.g. breathing, organs of perception and action). In the Attentiveness Level we use the Concentrating Modality in synchronizing higher level Subprocesses including the mental Ego faculty subprocesses, and the Awareness Level uses the Meditating modality in synchronizing higher level Subprocesses including the mental Mind faculty subprocesses. Finally in Wholeness Level is used by the EFS when the entire framework is synchronized as a whole.

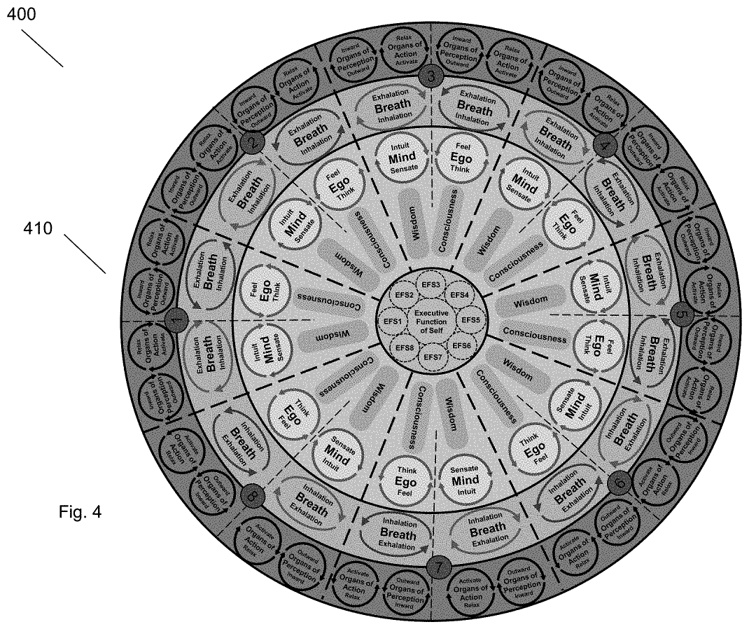

FIG. 4 Unit 410 illustrates in more detail the novel representation of Unit 320, whereby the angular axis is segmented into a set of eight sequenced, angular and generic experiential States (and respective Transitions between such States), comprising a complete and generic Process cycle that continually recurs over time. Each State comprises (i) Subprocesses of the organs of perception and organs of action of the physical sheath, (ii) Subprocesses of breathing of the energy sheath, (iii) Subprocesses of the volitional Ego faculty of the sheath of Consciousness, (iv) Subprocesses of the spontaneous Mind faculty of the sheath of Wisdom, and (v) Subprocesses of the EFS of the sheath of Bliss.

Below are the basic eight States of the Experiential Process.

| | The Eight | The Eight States |
|---|---|---|
| | Experiential States | of Presence |
| # | (Activation) | (Contemplation) |
| 1 | Intention | Purpose |
| 2 | Learning | Insight |
| 3 | Choosing | Values |
| 4 | Action | Integrity |
| 5 | Creation | Realization |
| 6 | Gratification | Gratitude |
| 7 | Restraint | Containment |
| 8 | Release | Freedom |

Experiential Process durations typically may well range from momentary (less than a minute), brief (minutes), short (hours), medium (days-months), long (years) to lifelong.

Further on the structure of the full Construct model (as illustrated in FIG. 4) and reference Process follows including a State-by-State description:

State #1: While consciousness and its modalities pervade the sheath of wisdom, the mental sheath is the vehicle of the proposed cyclical experiential Process of consciousness and existence. A cycle's actuality commences with the mental sheath entering the first State of a Process, turning inwards to the respective experiential Essence of Duty reservoir for arousal and inspiration, and activating a conscious Mindstate motivating of Will, followed by an Ego State manifestation of the motivating Egostate of Intention. As Mind Faculty activation precedes the Ego faculty (in each State), so does the Mindstate of Will precede the Egostate of Intention in this first State. A sincere sense of Egoic Intention cannot arise without a sense of Mindful Will that is aroused and motivated by a deep sense of Duty. Without the motivating Egostate of Intention, a constructive cyclical Process cannot commence.

State #2: The mental sheath cycle Transitions next to the second State of Learning, and upon entering this State turns inwards to the experiential Essence of Wisdom reservoir for energization and guidance. As the Mind faculty precedes the Ego Faculty, so is the perceptual Mindstate of Reverence a pre-requisite to any such newly acquired cognitive Egostate of Learning. Sincere respect and awe of knowledge and the practical learning process is a necessity for a skillful, creative and enlightening cycle. This State naturally persists until sufficient knowledge is acquired by Egoic Learning for a subsequent and requisite Choice to be made upon transitioning to the third mental sheath State of Choice.

State #3: The fundamental utility of the previously learned knowledge is the capacity to make the right and conscientious choice by the forthcoming Egostate of Choice. Upon the entering to this State, one turns inwards to the essence of Morality for virtuous arousal and moral guidance that activates the Mindstate of Conscience, underlying and facilitating subsequent Egoic Choice. A conscientious Ego-driven Choice is a fundamental motivating Egostate of any process, naturally followed by selective Action in the next State that best manifests the Choice made.

State #4: As the mental Process proceeds to the next State of Action where actual work is to be performed (based on the choice taken), one turns inwards to the Essence of Love, the Love for humanity and the admiration of nature. This in turn activates the Mindstate motivator of Compassion, a sense that best directs and guides any subsequent Ego-derived Action. A Compassion-driven Egoic Action is a fundament in any cycle, as it is requisite to the cycle's subsequent Creation. Throughout the State of Action, change and newly created outcomes are generated, enabling manifest circumstances and situations to evolve. Upon consummation of Action, the first extravertive half-phase of the cycle is complete.

Note: The ending of manifest Action is also the outset of the introvertive second half of the cycle. Although all Essences are radially energizing, some are directed outwards while others are directed inwards. While the first four Essences of the States, namely Duty, Wisdom, Morality and Love are naturally associated with outgoing and extroverted actuality, the remaining four Essences namely Bliss, Joy, Peace and Freedom are associated with the ingoing, introverted potentiality.

State #5: Upon the consummation of Action and a resulting manifested change, the experiential Process transitions to the next State, and turns inwards to the Essence of Bliss, seeking and revealing an ability to transcend in exhilaration beyond mere existence, that is experienced as a motivating Mindstate of Revelation that presets the motivational Egostate of Creation. Creation is the Ego's practical underscoring of meaning of the first half cycle's Action, and bears meaningfulness and enlightenment that are the manifests of the Essence of Bliss.

State #6: Following the heightening, albeit timely experience of blissful Creation, there follows a sense of simmering and internalization, whereby one turns inwards to the Essence of Joy for a deep sense of gratification, whereby a motivating Mindstate of Realization is experienced, followed by motivating Egostate sense of Contentment.

State #7: Following further with the Process, the experiential Process transitions into the next State and turns further inwards to the Essence of Peace, seeking internal self-control, balance and peacefulness upon the modest realization of the inherent limitations of any such Creation. This is the basis for developing a motivating Mindstate of reconciliation and acceptance, followed by a motivating Egostate of temperance, self-control and restraint are called upon, guided and energized by the Essence of Peace. Inner Peace is manifested by a sense that we are not more than human, and our creation, however inspiring, bears our natural limitations.

State #8: Upon the experience of inner Peace, the experiential Process Transitions to the eighth and final State of the cycle, and turns inwards to the Essence of Freedom, seeking the deepest sense of internal release and an end of the introvertive second half of the cycle's, thereby enabling an eventual onset of a new cycle. The transitioning to this State first activates a motivating Mindstate of Containment, followed by the activation of the Egostate of Surrender and a deep sensation of internal liberation. Containment is the mental absorption of all that was experienced throughout the entire Process into (conscious and unconscious) memory. The entirety of each cycle and its experiences are absorbed and retained, and nothing is left out nor overlooked. The Egostate of Surrender, brief and timely as it may be, is a release of consciousness, thereby freeing such internal mental and energetic space for a forthcoming commencement of the next cycle of a Process and its first State.

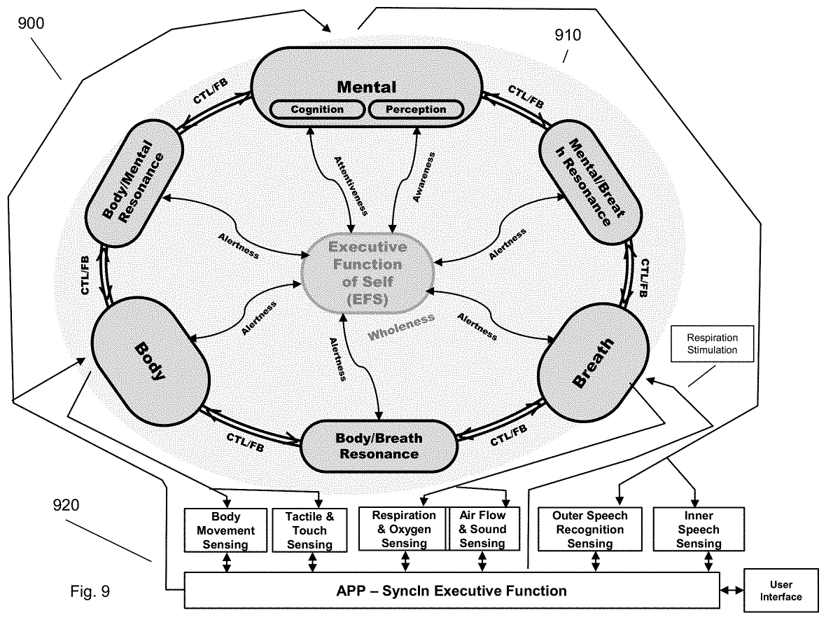

From ancient times, mandalas have been used worldwide in various spiritual traditions and cultures. The experiential Construct in FIG. 9 is designed in a graphical form of a mandala, that encompasses a comprehensive functional and circular mapping of the entire human framework, and in turn supports a generic cyclical Process as postulated by this treatise. A mandala is generally defined as a circular graphic design representation of wholeness and divinity that reflects upon us, inspiring spiritual growth and self-development in all aspects and circles of life.

Once synchronization of all the Subprocesses takes place, a sense of continuum of a Wholeness is experienced and this associated with inner Bliss as provided by ancient Yogic philosophy. In this highest Level of Oneness we use the Unifying Modality to unify the eight States of an entire Process into one continuum. In this highest Level, a sense of infinite internal and external continuum is experienced, where one experiences a deep sense of liberation, freedom and becoming one with nature. This diffuses mental attachments and blockages, and connects one with the entire functional map of the cyclical Process, enhancing our inner sense of existence, and a sense of Being in the now.

In the full experiential Construct of Unit 410, the notation of cyclical successions is such that proceeding forward transitions are clockwise, while receding backward transitions are counterclockwise. Moreover, forward transitions as well as backward transitions (and combinations thereof) are possible, depending ontcircumstance, experiential context and energy levels. Intermittent recurrences of such Transitions (within a State and across adjoining States) are called Nutations.

With a proceeding succession of transitions into a subsequent State of the cycle, the Mind faculty is first activated by performing spontaneous sensating and intuiting functions, and preferably synchronized with respective breath cycles. The eventual transition to the Ego faculty, is achieved by pulling away from the Mind subprocess, and flowing onto a respective Ego subprocess within that State, as the Ego engages the volitional thinking and feeling functions. Receding successions follow similarly, only in a backward direction.

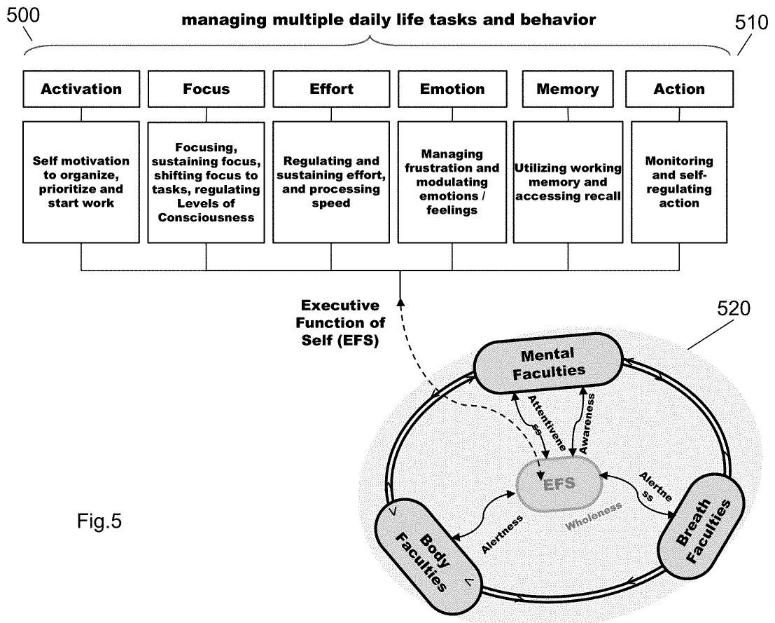

FIG. 5 Unit 510 presents the EFS behavioral functionalities as provided in current cognitive and behavioral science literature, and Unit 520 illustrates a functional flow of the EFS and BBM faculties in accordance with this invention.

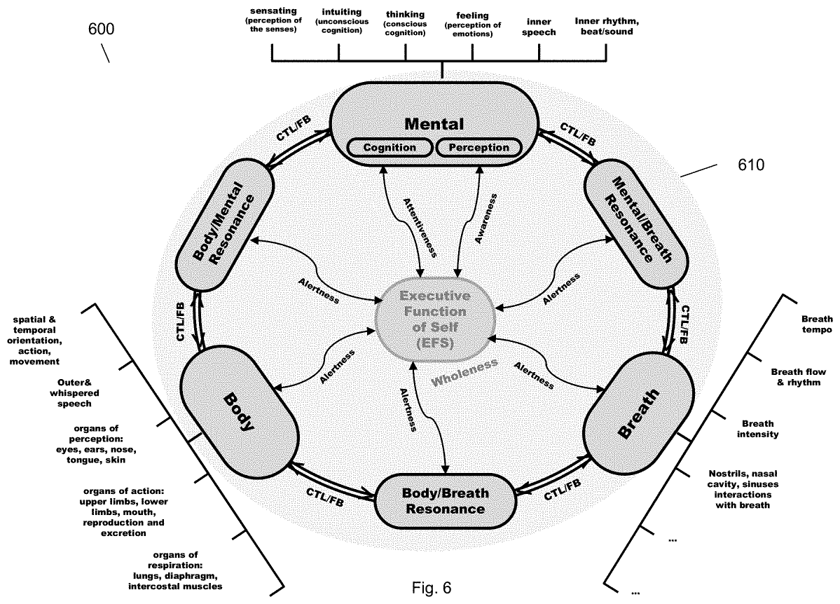

FIG. 6 Unit 610 illustrates a preferred representation of the framework of the EFS, and which establishes the need to specifically exercise the modalities of Alertness, Attentiveness and Awareness. In this representation, the outer control and feedback Alertness loop (high frequency, short response time) connects the respective BBM faculties and does not require the continuous control of the EFS. In this configuration, the EFS interacts (i) thru the slower and timely Attentiveness channels with the volitional functions of the Mental faculty, and (ii) thru the observant Awareness channels with the spontaneous functions of the Mental faculty. In this structure, Alertness interaction can be rapid as necessary, while Attentiveness interactions can be slower and timely, and the Awareness interactions may be slower in observing the other interactions as they evolve.

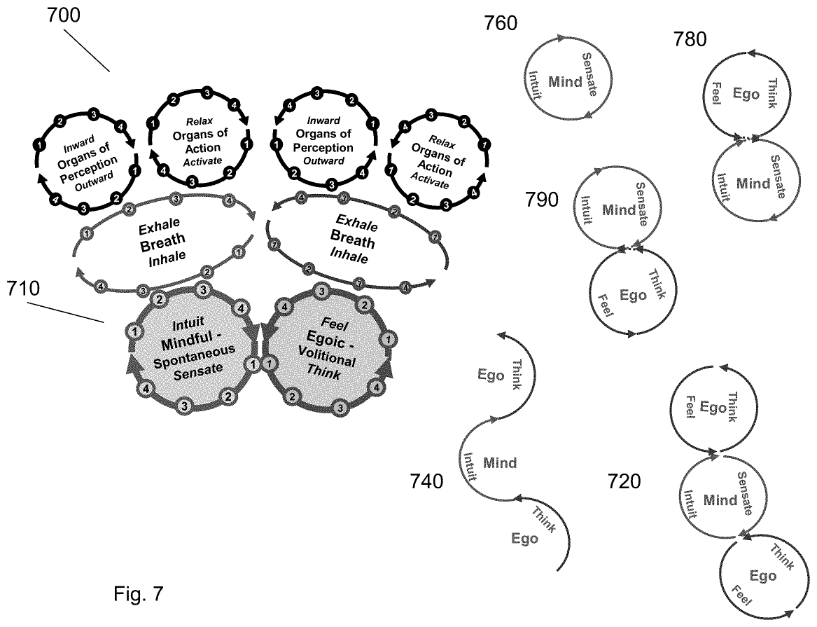

FIG. 7 Unit 710 illustrates for clarity Units 120,130 in the geometry as appearing in Unit 410, of synchronized transitions of the faculties. Units 720-790 further depict examples of Mind and Ego subprocess transitions and nutations following the arrows of respective functions. Unit 720 illustrates transitions within and between Ego, Mind, Ego-to-Mind and Mind-to-Ego faculties. Unit 740 illustrates an Egoic Think function transition to a Mindful Intuit function which then Transitions to another Egoic Think function. Unit 760 illustrates a Mindful nutation between the Sensate and Inuit functions. Units 780 and 790 illustrate nutations between adjacent Mind and Ego faculties.

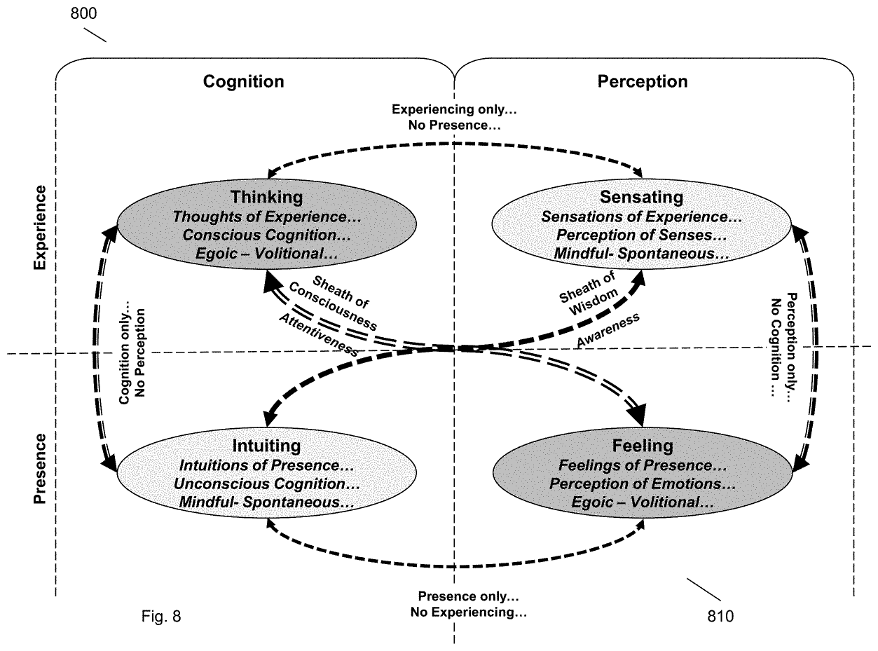

FIG. 8 Unit 810 illustrates the four mental functions (thinking, feeling, sensating and intuiting), and their categorizations in terms of (i) cognition and perception and (ii) experience and presence, in accordance with this invention. It is noted that different mental function transitions are associated with specific sheaths and modalities.

In exercising the MBB EFS model of FIG. 6, a man-machine emulation of the model is proposed in FIG. 9 Unit 910, in a form that facilitates a computerized implementation that can be configured, for example, as a mobile APP or a desktop software program. In Unit 920 the EFS model is emulated by a SyncIn Executive Function (SEF) that generates exercise sequences, beat and rhythm patterns, graphic displays and sounds generation, as well as sensing provisions of, for example, body movement, tactile and touch instances, breath and respiration, air flow and sounds, outer and inner speech.

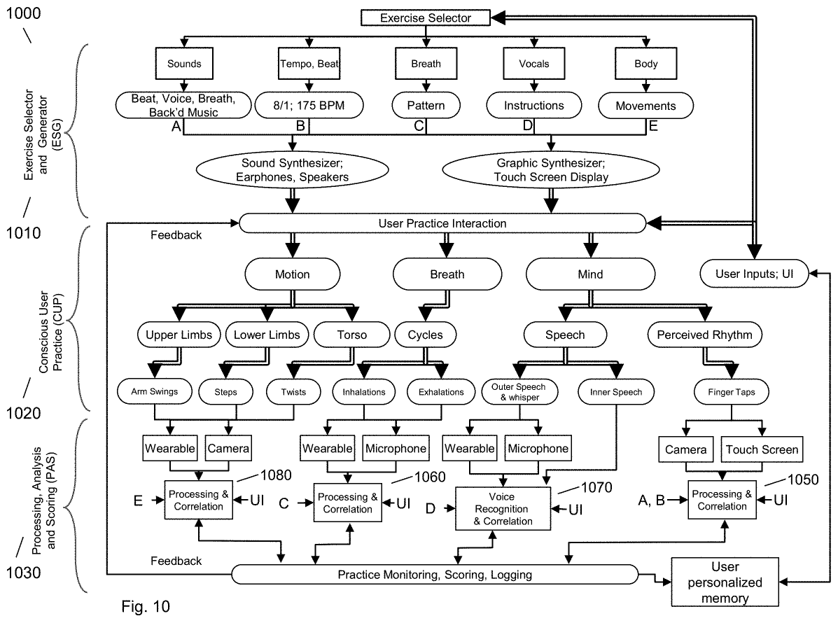

FIG. 10 Unit 1000 illustrates the functional layout and conceptual design of the SyncIn exercise APP, comprising three main processes namely: (a) Unit 1010—Exercise Selector and Generator (ESG), (b) Unit 1020—Conscious User Practice (CUP) and (c) Unit 1030—Processing, Analysis and Scoring (PAS).

The ESG composes the user-selected exercise through a sounds function output A, tempo/beat function output B, breath function output C, vocals function output D and body function output E. These function outputs generate sounds thru earphones and speakers, interactive graphics display and touch screen interaction. All these can be generated with for example a mobile device, a desktop application etc. The CUP comprises the user practice interaction that responds with conscious body motion, conscious breath and conscious mind (mental) interaction. Without limitation, the body motion comprises upper limb motion (arm swings), lower limb motion (footsteps) and torso motion (twists and extensions), while breath comprises inhalations and exhalations, and mental functions comprise outer speech, whispering and inner speech, as well as perceived rhythm (thumbs, finger tips). These are sensed and processed by the PAS.

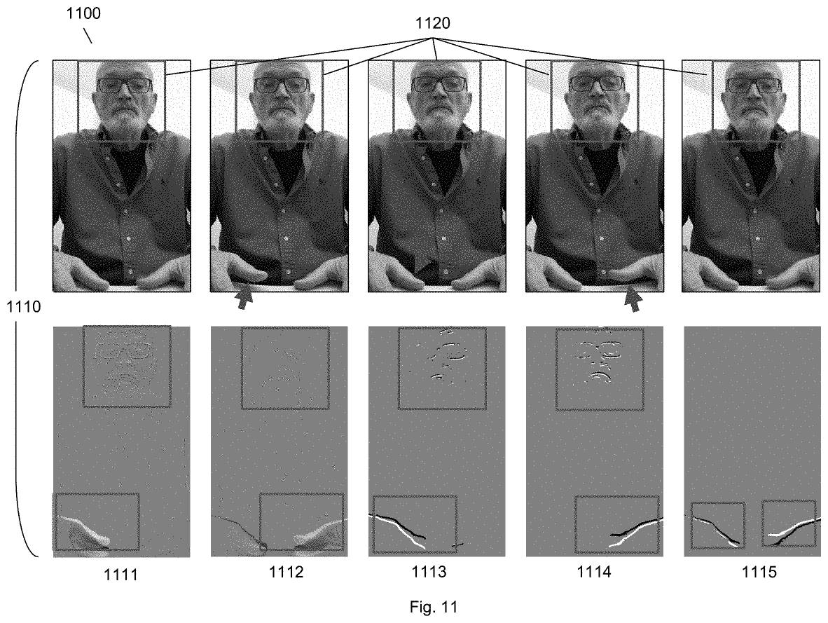

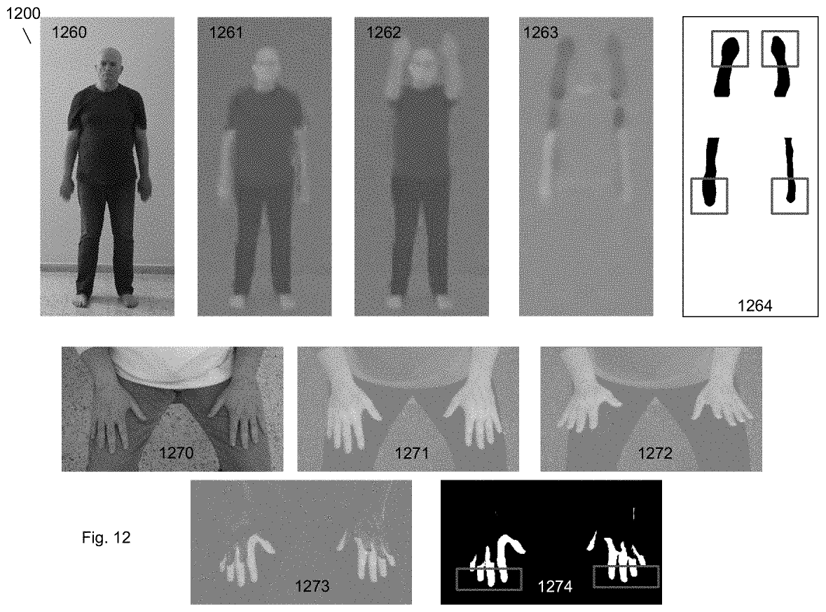

A preferred embodiment of PAS Unit 1030 in this invention applies multiple measurements and processing to the A,B,C,D,E modalities in analyzing and scoring the exercises as performed by the user, and in providing on-line and off-line feedback to the user in further improving his/her practice for improved alertness, attentiveness and awareness, thereby facilitating improved wellness. A preferred embodiment of assessing perceived rhythm by the user based on thumb and finger tapping (as well as lower and upper limb motion and torso motion) is sensed and measured in Unit 1050 utilizing a camera (e.g. mobile video camera) and/or the touch screen function on which rhythmic thumbs and/or fingers tapped (e.g. mobile touch screen) in measuring the perceived rhythm of the user, and correlative processing comparison and scoring of the actual tapping to the intended exercise rhythm and/or background music. Another preferred embodiment of same is utilizing a camera and applying machine vision algorithms of detection and tracking of thumbs/fingers as illustrated in FIG. 11 Unit 1100. Units 1111-2 processing output preferably perform a region-of-interest (ROI) allocation operation detecting roughly thumb/finger motion applying spatio-temporal differencing, thresholding and blobbing of right hand and left hand, respectively, and Units 1113-4 processing output preferably perform a tracking and contour delineation of the moving thumb/finger in determining the direction (i.e. up or down) of the moving thumb/finger. Units 1120 are facial detection/recognition outputs of the user during exercise, used to associate user identity to exercise and practice usage. Unit 1115 combines all results in a unified scene representation. The output of the facial recognition results may be used by health providers and health insurance companies to compensate users accounting for their improve wellness practice effort and results that reduce overall health care costs.

The mind-body interpretation of perceived rhythm onto lower and upper limb motion and torso motion, as sensed and measured in Unit 1080 utilizing a camera (e.g. mobile video camera) and/or a wearable device in measuring the perceived rhythmic motion of the user, and correlative processing comparison and scoring of the actual motion to the intended exercise rhythm and/or background music.

Unit 1260 depicts an erect standing, dual-arm-swing exercise opening position, and Units 1261-2 depict a preferred embodiment output of the scene following an RGB color enhancement function that accentuates the skin tone contrast of the user as compared to the background, using an image processing image-differencing function Red-Green or Red-Blue (R-G, R-B). Units 1263-4 processing outputs preferably perform an operation detecting roughly hand/arm motion by applying spatio-temporal differencing, thresholding and blobbing of right and left hand/arm and a ROI allocations of extreme (distal) parts of the said hands/arms, hence providing synchronization accuracy of actual body parts motion compared to the intended exercise rhythm and/or background music. Similar processing step outputs are illustrated by Units 1270-1274 for a seated exercise and body motion of finger taps.

The mind-breath interpretation of perceived rhythm onto inhalation and exhalation cycles, as sensed and measured in Unit 1060 utilizing a microphone (e.g. mobile microphone) and/or a wearable device in measuring the rhythmic airflow of breath cycles and correlative processing comparison and scoring of the actual breath to the intended exercise breath rhythm, comprise a key element of this invention.

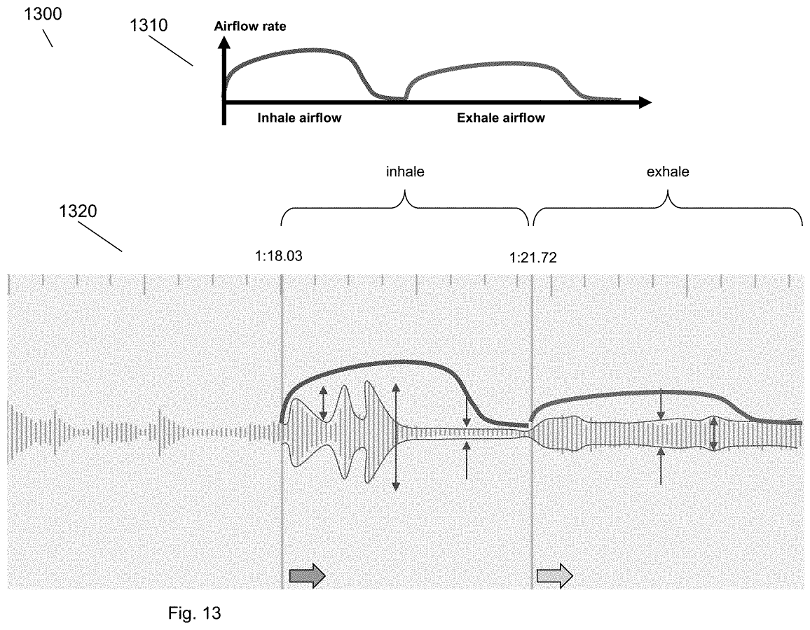

Unit 1300 in FIG. 13 illustrates an exemplary (nasal) airflow sound-based time plot of a breath inhalation/exhalation cycle using for example, a mobile device microphone. Unit 1310 illustrates a nominal inhale/exhale airflow time plot characteristic with minimal airflow abruptions, flow variations and a certain balance between inhalation and exhalation airflow. Unit 1320 is a comparable plot with prevalent variability over time. Hence such a measurement may be correlated with nominal desired breath cycles in terms of variability as well as synchronization with exercise-defined breath rates. Such comparison and correlation may be used to feedback improvement cues in real time during exercise as well off-line.

The mind-speech interpretation of perceived rhythm of outer speech and whispered-speech, as sensed and measured in Unit 1070 utilizing a microphone (e.g. mobile microphone) and/or a wearable device in applying voice recognition processing algorithms and correlative processing comparison and scoring of the actual speech instructions and patterns to the intended exercise patterns, comprise another key element of this invention. Inner (silent) speech cannot be directly measured or sensed; However, by interleaving outer-speech (or whispered-speech) with (silent) inner-speech thereby overcoming this limitation and which comprises another key element of this invention.

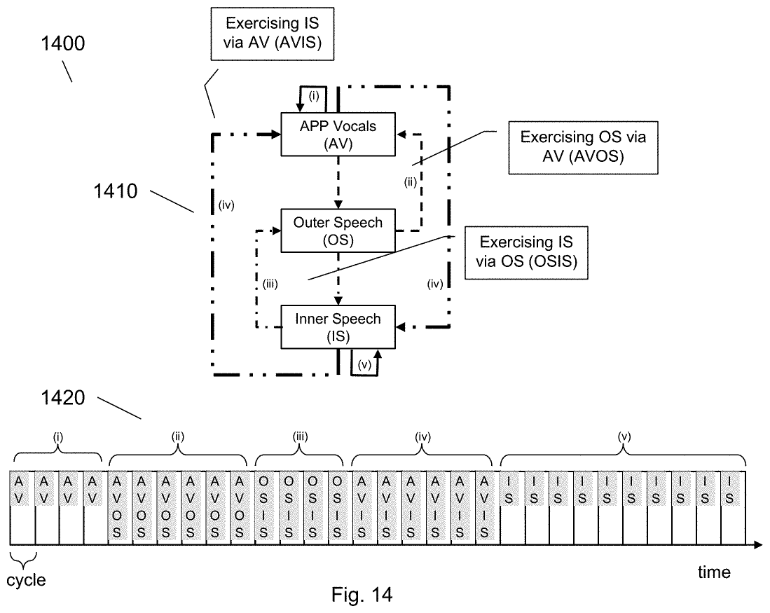

Unit 1400 in FIG. 14 illustrates a preferred embodiment of an exemplary method that progressively interleaves outer-speech (or whispered-speech) with (silent) inner-speech thereby facilitating the exercising of outer-speech, whispered-speech combined with inner-speech. Unit 1410 illustrates a progressive method that facilitates a learning and training process of outer- and inner-speech starting with APP vocals that are progressively replaced by outer-speech, and eventually by inner-speech, as better illustrated in Unit 4720, and described in detail in the following progressive exercise stages:
(i) AV: Using SyncIn APP-generated Vocals (AV) while exercising. This is the entry stage, where the user is not familiar with exercise sequences and emphasis is put on simply carrying out the vocal instructions generated by the APP using earphones or a speaker.
(ii) AVOS: Using AV in exercising Outer Speech (OS), whereby AV and OS are temporally interleaved thereby allowing the user to slowly learn and exercise the SyncIn exercise outer-speech/vocal instructions, while the APP is able to apply OS voice recognition in scoring and providing feedback to the user.
(iii) OSIS: Using OS in exercising Inner Speech (IS), whereby OS and IS are temporally interleaved thereby allowing the user to slowly learn and exercise the SyncIn exercise Inner-speech/vocal instructions, while the APP is able to apply OS voice recognition in scoring and providing feedback to the user.
(iv) AVIS: Using AV in exercising IS, without the ability to apply voice recognition, however able to analyze motion, rhythm and breath synchronization thereby providing a partial level of analysis on the effect of inner speech.
(v) IS: Using IS only in exercising SyncIn, once sufficiently trained in diverse exercises.
   By such periodical interleaving of the above exercise stages, the user proficiency is progressively improved, while the APP continuously analyzes accuracy of synchronicity of the different modalities even when eventually the user utilizes only silent inner-speech.

In the context of this invention, wellness is associated with the homeostasis thru synchronization of the BBM faculties, and therefore the means and method of such synchronization are a key objective and element of this invention, whereby each such faculty is based on differing principles, i.e. body on physiology, breath on energy and mental on cognitive and perceptual processes. While breath is inherently repetitive, body and mind are not, therefore requiring specific methodology and means (i.e. technology, algorithms) for such timely synchronization that comprises an essential element of this invention.

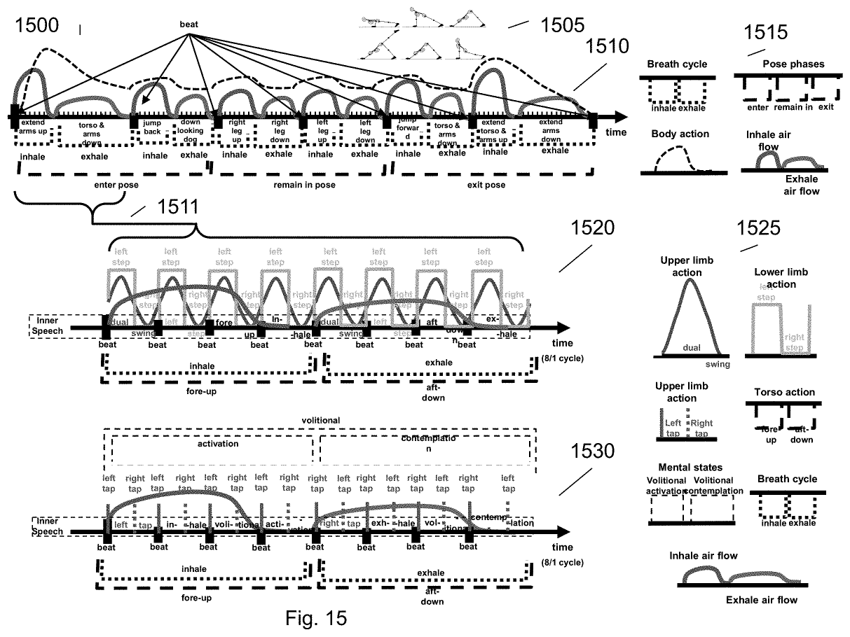

FIG. 15 Units 1510, 1520 and 1530 depict exemplary time plots of combined mindful movement/breathing exercises that are compared in terms of their levels of synchronization of the BBM faculties, comprising respectively elements depicted in Units 1515 and 1525. Specifically, Unit 1510 depicts a time plot of a well-known exemplary mindful movement Yoga pose, namely “down looking dog” Unit 1505 comprising three pose stages including an enter pose stage, a remain-in (maintain) pose stage and an exit pose stage. Note that neither body action nor the time beats are synchronized with breath cycles in the enter and exit stages of the pose, and while offering many associated benefits, it does not synchronize the BBM as provided in the proposed exercise methodology and exercise monitoring technology for improved wellness as provided in this invention. Unit 1511 illustrates an exemplary structure of synchronized BBM exercise actions for each single breath cycle as provided in Unit 1520 comprising an inhalation and exhalation that all are fully synchronized to beats (in an exemplary 4:4 pattern), including standing (a) upper limb action, (b) lower limb action, (c) torso action, (d) breath cycles and (e) mental inner speech that synchronously, internally and textually dictates the actual respective actions taken, in this exemplary case during inhalation “dual/swing-left/step-fore/up-in/hale” followed during exhalation “dual/swing-left/step-aft/down-ex/hale” (“/” is at half beat, “-” is at full beat).

Another exemplary standing, seated or supine exercise as provided in the proposed methodology is depicted in Unit 1530, whereby in addition to synchronized breathing cycles with beats (in an exemplary 4:4 pattern), synchronized body action is hand tapping along with mental volitional activation (during inhalations) and contemplation (during exhalations). Inner speech of the experiential inner reflection points in this exemplary case is during inhalation “left/tap-in/hale-voli/tional-acti/vation” followed during exhalation “right/tap-ex/hale-voli/tional-contemp/lation” (“/” is at half beat, “-” is at full beat). Generally desired beat rates, rhythm patterns and sounds may be imagined or sounded during exercise. The mental exercise of the inner speech (or whispering) of the experiential inner reflection points is the anchor of such synchrony of the BBM.

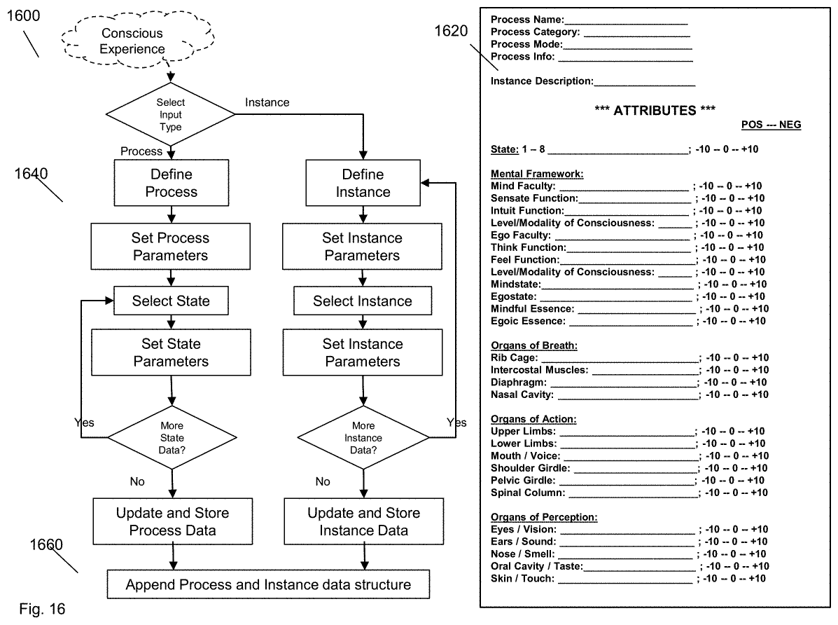

FIG. 16 Unit 1620 illustrates an exemplary representation of an experiential instance of the mental framework at time T, in accordance with this invention:

I_{T}(S₁(A),S₂(A),S₃(A),S₄(A),S₅(A),S₆(A),S₇(A),S₈(A)) (Eq #1)

where Sₓ is State #X, and A represents the values of the attributes vector above

A=(MF,SF,IF,EF,TF,FF,MS,ES,ME,EE,LMC) (Eq #2)

with multiple features' attribute values ranging (for example) between −10 to +10.

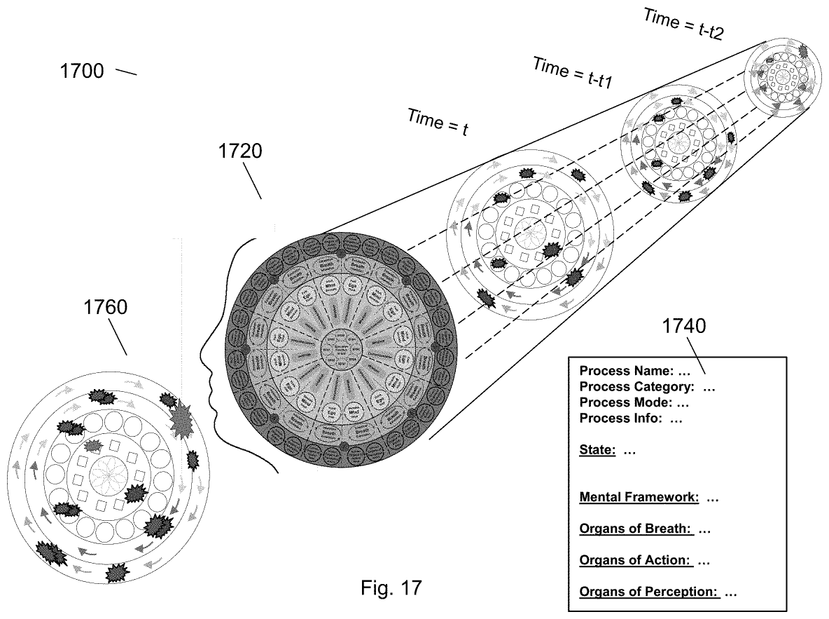

An experiential Instance I_{T} may include at least one State for which at least one feature attribute value is given. Such Instances experienced at different times T may be accumulated as described below (FIG. 17).

FIG. 17 Unit 1720 presents a flow diagram that describes experiential Instance information accumulated over time (T=t, T=t−t1, T=t−t2 etc.) and stored in an accumulated Construct structure, and further logged in data structure Unit 1760. The coming into being of a Process based on accumulating experiential Instances over time is another key element of this invention, as the temporal behavior and buildup of a Process reflects an important characteristic. For example, a belief takes time to buildup, and is often based on accumulating experiential Instances all of which comprise and are part of an evolving Process. As more and more experiential Instances are accumulated in a given Process, a statistical analysis may be applied to each feature's attribute values and statistical parameters may be derived.

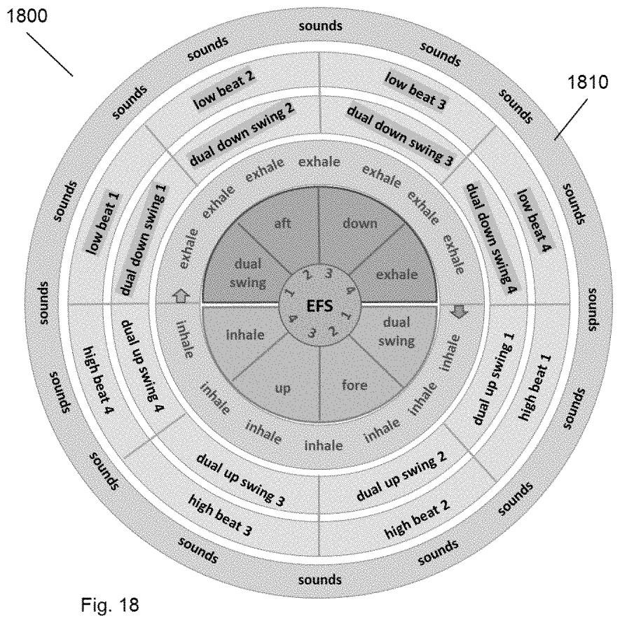

FIG. 18 illustrates an exemplary circular concentric representation of a basic SyncIn mindful MV exercise, in accordance with this invention. Unit 1810 depicts the respective experiential cyclical and repetitive model of BBM represented by several concentric circles, all revolving clockwise synchronously, as provided in this invention, and facilitating conscious exercise and progressive experiential practice using preferably a computerized software program (e.g mobile APP or desktop software program), thereby raising one's awareness to his/her inner works and continually evolving inner change. In Unit 1810 an exemplary 4:4 BBM exercise with eight (1-8) numbered steps, where the first four steps are associated with inhalation and the last four steps are associated with exhalation (inner green circle). The outer circle (green) represents background sounds (e.g. music) that may be played during exercising. The next inner circle (yellow) represents and plays the beat where for example, the first four steps in inhalation are sounded as synchronized high tone beats, and the last four steps in exhalation are sounded as synchronized lower tone beats, thereby providing a sound cue and facilitating breath synchronization during practice. The next inner (yellow) circle represents in this example upper limb action such as dual up swing which represents synchronized dual arm swings that while inhalation the hands are facing upwards and during exhalation and dual arm swings hands are facing downward. The next inner circle (red during inhalation and blue during exhalation) represents the inner speech dictating the actual actions of the exercise; In this example, during the inhalation and the first four beats and steps, the inner speech dictation is “dual/swing-fore-up-inhale”, during the exhalation and the last four beats and States, the inner speech dictation is “dual/swing-aft-down-exhale”, where the “for/up” and “aft/down” represent front/rear body orientation and the up/down direction of breath and facing directions of the hands. In this manner, BBM are focused and holistically synchronized.

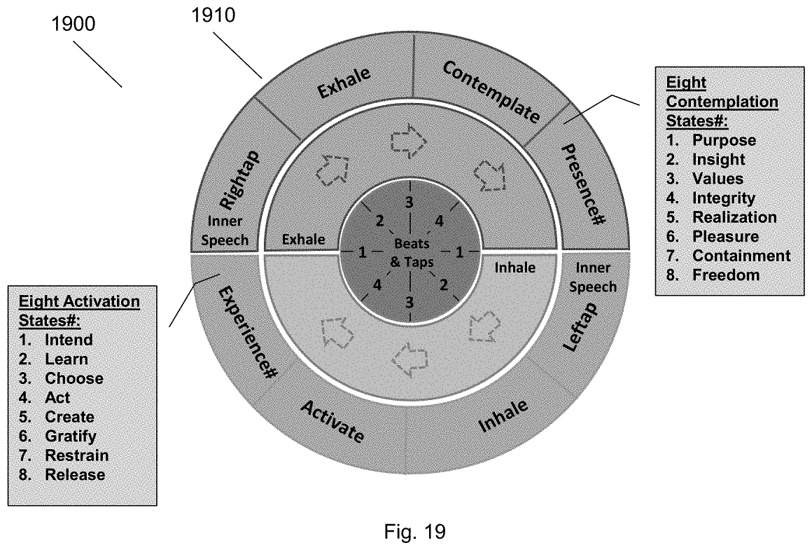

Similarly, FIG. 19 Unit 1910 depicts tapping as action rather than arm swings, and the inner speech dictating eight subsequent inner reflection points of the experiential Construct associated with volitional attentiveness, namely activations during inhalations and contemplations during exhalations. The innermost (brown) circle now represents hand tapping action. The next outer circle (red during inhalation and blue during exhalation), and outermost circle represents the inner speech oration of the actual actions and inner reflection points of the exercise; In this example, during the inhalation and the first four beats and steps, the inner speech dictation is “lefttap-inhale-activate-experience #”, during the exhalation and the last four beats and steps, the inner speech oration is “rightap-exhale-contemplate-presence #”, where the “#” represents the respective index of the activation/contemplation of the volitional attentiveness State. In this manner, BBM are focused and holistically synchronized also with respect to the volitional attentiveness aspect of the experiential Construct

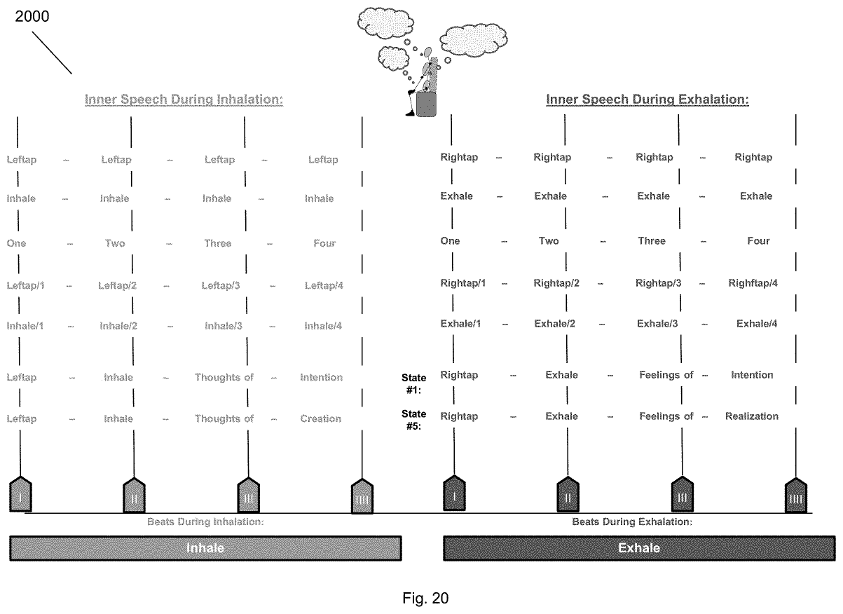

FIG. 20 Unit 2000 illustrates an exemplary set of inner speech sequences, whereby the first five exercises aim to train the novice, and the last two sequences relate to State$1 and State 5.

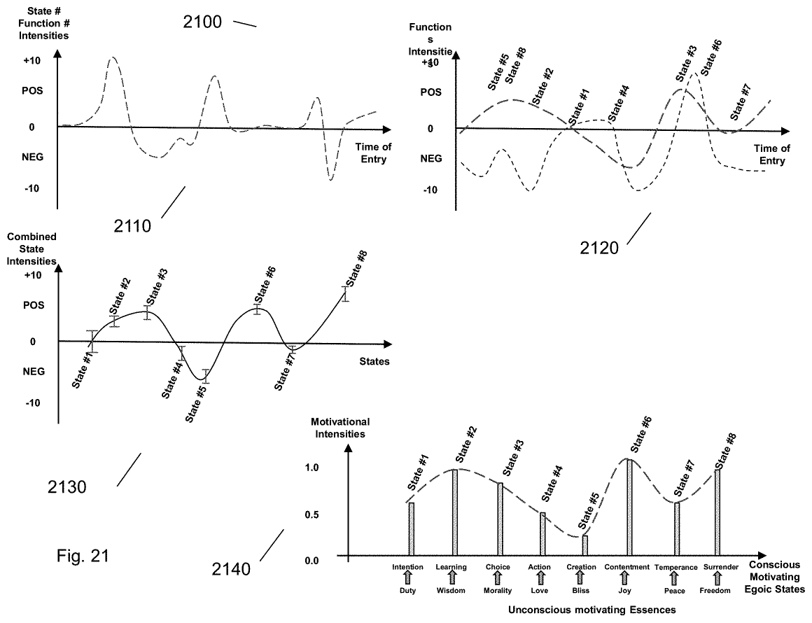

FIG. 21 Units 2120, 2130, 2140 illustrate various one-dimensional to n-dimensional graphical representations of computed State, Function and attribute statistics, in accordance with this invention, useful in the display of outcomes of such classifiers, as well as means of displaying broad experiential indications to a professional or non-professional user.

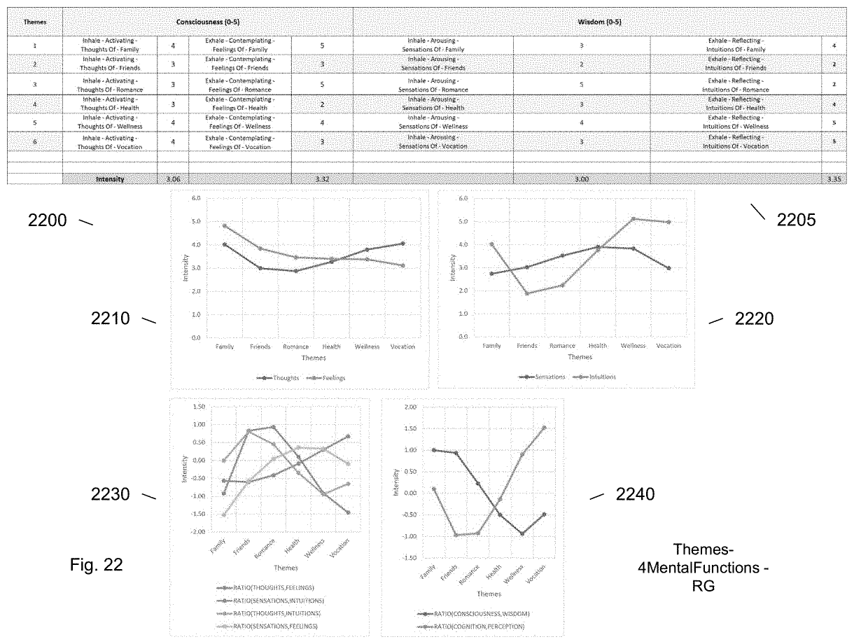

FIG. 22 illustrates exemplary user entries of intensities using MD inner speech exercises that activate queries using the four mental Functions in pairs: (a) dual Thoughts-Feelings inner speech sequences and (b) dual Sensations-Intuitions inner speech sequences, and whereby six generalized themes that are meditated upon are of general interest to all, and which reflect our personality traits, in accordance with this invention.

The exemplary inner speech meditative sequence is used to describe the computational process:

| Themes | Consciousness (0-5) | | | | Wisdom (0-5) | | | |
|---|---|---|---|---|---|---|---|---|
| 1 | Inhale - Activating - | 4 | Exhale - | 5 | Inhale - Arousing - | 3 | Exhale - | 4 |
| | Thoughts Of - | | Contemplating - | | Sensations Of - | | Reflecting - | |
| | Family | | Feelings Of - | | Family | | Intuitions Of - | |
| | | | Family | | | | Family | |
| 2 | Inhale - Activating - | 3 | Exhale - | 3 | Inhale - Arousing - | 2 | Exhale - | 2 |
| | Thoughts Of - | | Contemplating - | | Sensations Of - | | Reflecting - | |
| | Friends | | Feelings Of - | | Friends | | Intuitions Of - | |
| | | | Friends | | | | Friends | |
| 3 | Inhale - Activating - | 3 | Exhale - | 5 | Inhale - Arousing - | 5 | Exhale - | 2 |
| | Thoughts Of - | | Contemplating - | | Sensations Of - | | Reflecting - | |
| | Romance | | Feelings Of - | | Romance | | Intuitions Of - | |
| | | | Romance | | | | Romance | |
| 4 | Inhale - Activating - | 3 | Exhale - | 2 | Inhale - Arousing - | 3 | Exhale - | 4 |
| | Thoughts Of - | | Contemplating - | | Sensations Of - | | Reflecting - | |
| | Health | | Feelings Of - | | Health | | Intuitions Of - | |
| | | | Health | | | | Health | |
| 5 | Inhale - Activating - | 4 | Exhale - | 4 | Inhale - Arousing - | 4 | Exhale - | 5 |
| | Thoughts Of - | | Contemplating - | | Sensations Of - | | Reflecting - | |
| | Wellness | | Feelings Of - | | Wellness | | Intuitions Of - | |
| | | | Wellness | | | | Wellness | |
| 6 | Inhale - Activating - | 4 | Exhale - | 3 | Inhale - Arousing - | 3 | Exhale - | 5 |
| | Thoughts Of - | | Contemplating - | | Sensations Of - | | Reflecting - | |
| | Vocation | | Feelings Of - | | Vocation | | Intuitions Of - | |
| | | | Vocation | | | | Vocation | |
| | Intensity | 3.06 | | 3.32 | | 3.00 | | 3.35 |

### Sheath of Consciousness: I-A-TO-F→E-C-FO-F

Inhale-Activating-Thoughts On-Family→Exhale-Contemplating-Feelings On-Family

In this example, the user-determined Experiential Intensity (EI) of the [I-A-TO-F] exercise is given by EI[T]=4, while the EI[F]=5. All values are plotted in Units 2210 and 2220. While both intensities are strong, the latter is stronger. A key element of this invention is the way the said EI(T,F) is derived from the two user intensities:

EI(T,F)=(EI[T]—EI[F])/(EI[T]+EI[F]))

RSS(EI[T],EI[F])=(EI²[T]+EI²[F])^{0.5}; {RSS stands for Root Sum of Squares}

RATIO(T,F)=EI(T,F)*RSS(EI[T],EI[F])

While EI(T,F) is the contrast measure between Thoughts and Feelings, the RATIO(T,F) represents a respective normalized contrast measure that is normalized to the power of the Thoughts and Feelings. Similarly, the same is repeated for the mental function dual pairs of (S,I), (T,I) and (S,F).

The dual pairs' RATIOS are plotted in Unit 2230 for all combinations of the four Mental Functions and Themes. In the present example, the exemplary Themes are: Family, Friends, Romance, Health, Wellness and Vocation. Additional Themes may include for example Intellectual Wealth, Financial Wealth etc.

A similar RATIO computation if performed for the contrast measures between Consciousness and Wisdom, and those between Cognition and Perception, and plotted in Unit 2240. For the above example:

EI(CON)=RSS(EI(T),EI(F))

EI(WIS)=RSS(EI(S),EI(I))

EI(CON,WIS)=(EI[CON]—EI[WIS])/(EI[CON]+EI[WIS]))

RSS(EI[CON],EI[WIS])=(EI²[CON]+EI²[WIS])^{0.5}

RATIO(CON,WIS)=EI(CON,WIS)*RSS(EI[CON],EI[WIS])

EI(COG)=RSS(EI(T),EI(I))

EI(PER)=RSS(EI(S),EI(F))

EI(COG,PER)=(EI[COG]—EI[PER])/(EI[COG]+EI[PER]))

RSS(EI[COG],EI[PER])=(EI²[COG]+EI²[PER])^{0.5}

RATIO(COG,PER)=EI(COG,PER)*RSS(EI[COG],EI[PER])

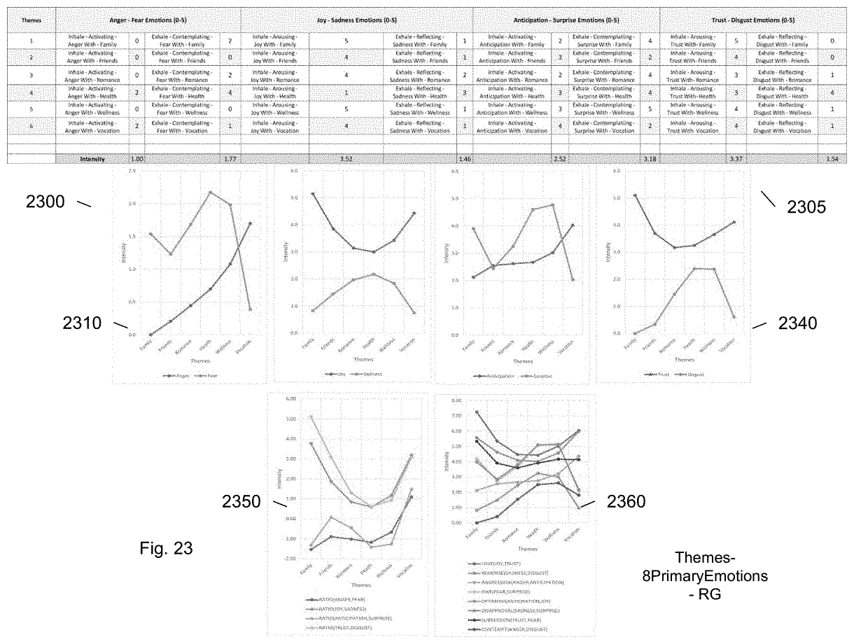

FIG. 23 illustrates exemplary user entries of intensities using MD inner speech exercises that activate experiential queries with respect to the selected themes (as in FIG. 22) using the eight basic emotions in dual pairs (Anger-Fear, Joy-Sadness, Anticipation-Surprise and Trust-Disgust, for each of the eight States of the Experiential Process, in accordance with this invention and plotted in Units 2310,20,30,40. The RATIO computations are performed similarly as done in FIG. 22 for the said basic emotion dual pairs and plotted in Unit 2350. Derived Feelings are computed and plotted in Unit 2360 where Feelings are defined as the RSS of combinations of basic emotion EI.

From a human behavioral perspective, the feeling and emotional aspects of the modeled experiential Construct are fundamental to the human paradigm, and to the Construct's utility and applicability in reflecting the experiential Processes of users. The emotional aspects of the human condition are a central affect in one's experiential Process, and are directly related to our driving motivational values. Emotions are considered by some theorists as discrete responses to internal or external events which have a particular significance for the organism. A neurobiological perspective is provided by Antonio Damasio, who distinguishes between emotion and feeling, whereby (i) an emotion is a “patterned collection of chemical and neural responses that the brain produces when it detects the presence of an emotionally competent stimulus”, and mentions specifically “fear, disgust, sadness, happiness, sympathy, love, shame and pride”, and (ii) a feeling is a “mental representation of the physiological changes” that were induced by the chemical and neural responses produced. He also suggests that one needs the perception of the changed bodily states “alongside the perception of a certain mode of thinking, and of thoughts with certain themes” to fully reflect a feeling of an emotion. The general form of the distinction is that the emotion itself is some neural/bodily event, and one feels that emotion when one perceives the bodily event. While emotions and feelings are fundaments of human existence, they are often difficult to describe, define and address.

A more practical approach follows the known art of Robert Plutchik who defines a map of emotions comprising eight basic emotions, four of which are opposites of the others: anger, anticipation, joy and trust, and their respective opposites namely fear, surprise, sadness and disgust. For each of the basic eight emotions, two additional associated emotions are designated, namely a milder one and a more intense one (as provided in Table #1).

TABLE 1

| Intense | Mild | Basic | Opposite | Mild | Intense |
|---|---|---|---|---|---|
| emotion | emotion | emotion | emotion | Opposite | Opposite |
| Rage | Annoyance | Anger | Fear | Apprehension | Terror |
| Vigilance | Interest | Anticipation | Surprise | Distraction | Amazement |
| Ecstasy | Serenity | Joy | Sadness | Pensiveness | Grief |
| Admiration | Acceptance | Trust | Disgust | Boredom | Loathing |

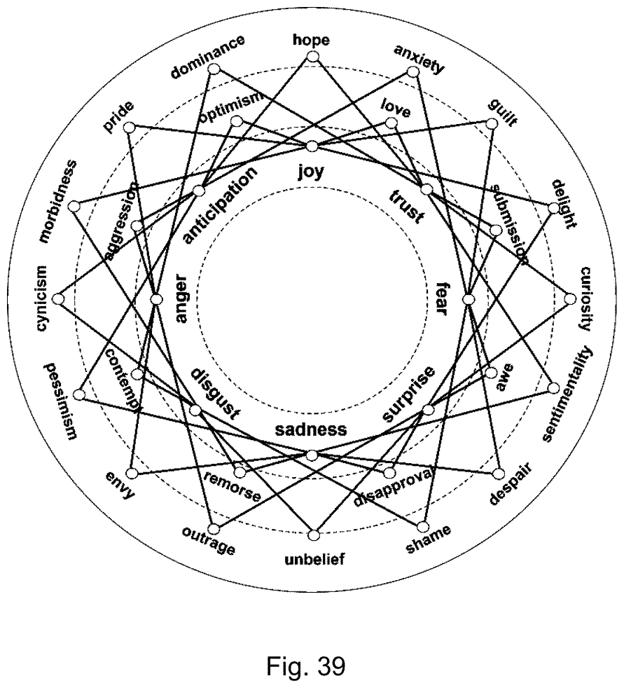

Plutchik also theorized that feelings are generally composed of two emotions, amounting to thirty-two feelings (including the eight emotions). A depiction of his wheel-like map is provided for reference in FIG. 39. This scheme is useful in defining the Feel Function attribute entries and their affect intensity, to best reflect the experiential emotional and feeling aspects of any instance.

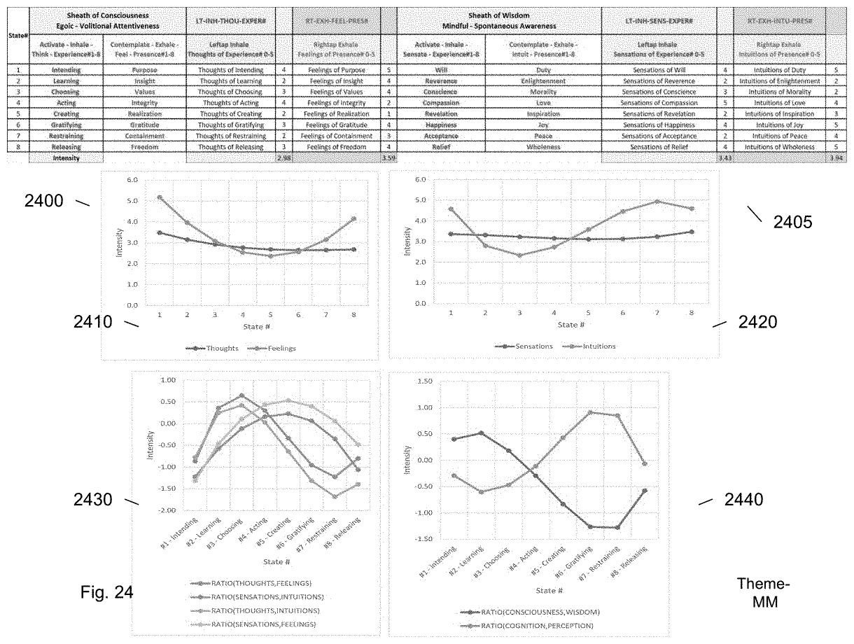

FIG. 24 illustrates exemplary user entries of intensities using MD inner speech exercises that activate queries using the four mental Functions in pairs: (a) dual Thoughts and Feelings inner speech sequences and (b) dual Sensations and Intuitions inner speech sequences, and whereby the eight generalized queries that are meditated upon are the eight States of the Experiential Process and the theme is related to a relationship, in accordance with this invention. RATIO computations are performed similarly to the FIG. 22 example, and plotted in Units 2430,2440.

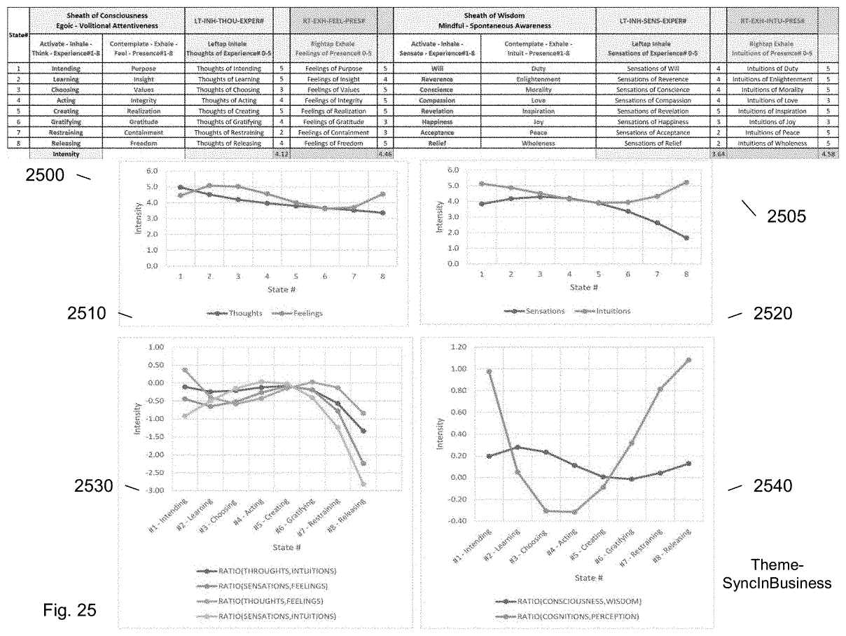

FIG. 25 illustrates exemplary user entries of intensities using MD inner speech exercises that activate queries using the four mental Functions in pairs: (a) dual Thoughts and Feelings inner speech sequences and (b) dual Sensations and Intuitions inner speech sequences, and whereby the eight generalized queries that are meditated upon are the eight States of the Experiential Process and the theme is related to the business aspects of a project, in accordance with this invention. RATIO computations are performed similarly to the FIG. 22 example, and plotted in Units 2530,2540.

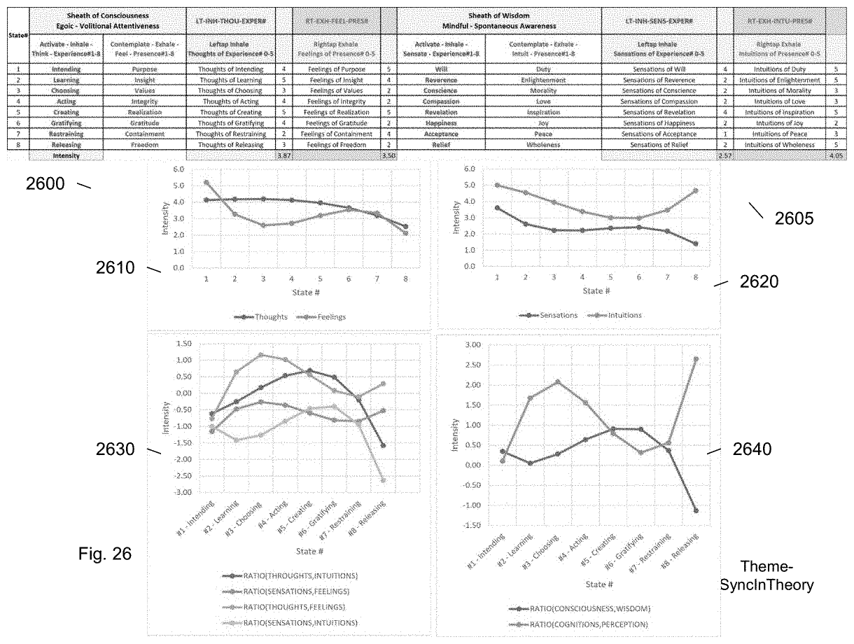

FIG. 26 illustrates exemplary user entries of intensities using MD inner speech exercises that activate queries using the four mental Functions in pairs: (a) dual Thoughts and Feelings inner speech sequences and (b) dual Sensations and Intuitions inner speech sequences, and whereby the eight generalized queries that are meditated upon are the eight States of the Experiential Process and the theme is related to the theoretical aspects of a project, in accordance with this invention. RATIO computations are performed similarly to the FIG. 22 example, and plotted in Units 2630,2640.

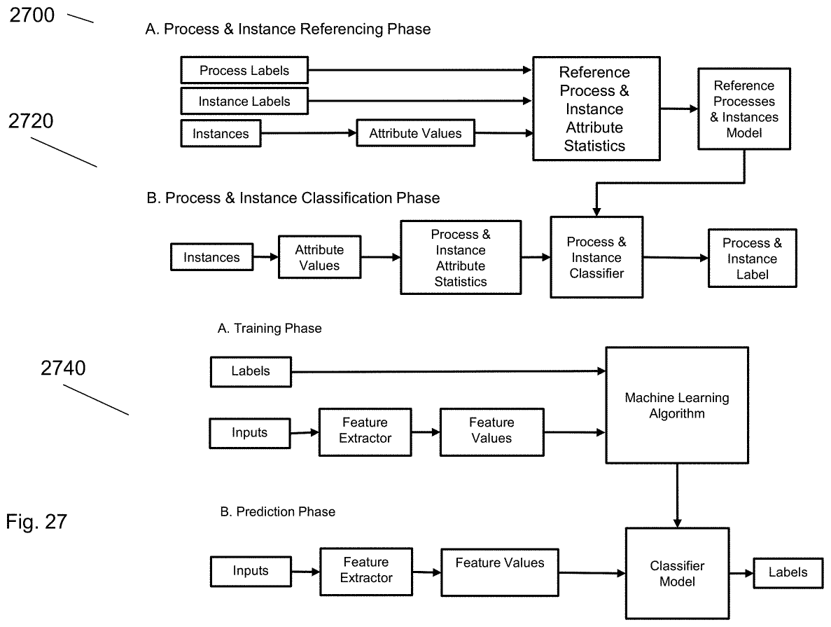

FIG. 27 illustrates two block diagrams of computational algorithms comprising (i) a Process and instance referencing phase and classification phase based on manually-entered experiential attributes in accordance with this invention, and (ii) a classical machine learning framework known in the art that combines a training phase and a prediction phase, in accordance with this invention. Unit 2720 (i) a machine learning framework comprising experiential Process and Instance referencing phase and classification phase based on manually-entered and/or technically-measured experiential attributes in accordance with this invention, and Unit 2740 (ii) a classical machine learning framework known in the art that combines a training phase and a prediction phase.

One preferred embodiment for Unit 2720 is a Naive Bayes Classifier well known in the art. It is a simple technique for constructing classifiers, as it assigns class labels to problem instances, represented as vectors of feature values, where the class labels are drawn from some finite set. Various algorithms are used to train the classifier, all based on a common principle: all naive Bayes classifiers assume that the value of a particular feature is independent of the value of any other feature, given the class variable.

For some types of probability models, naive Bayes classifiers are known to be efficiently trained in a supervised learning setting. In many practical applications, parameter estimation for naive Bayes models uses the method of maximum likelihood; in other words, one can work with the naive Bayes model without accepting Bayesian probability or using any Bayesian methods. An advantage of naive Bayes is that it only requires a small number of training data to estimate the parameters necessary for classification.

One preferred embodiment of Unit 2720 utilizes professionals such as therapists and psychologists, that have sufficient field knowledge to building labeled reference Instances and Processes including attribute values and variabilities. For example, for an Instance where State #1 is weakly motivated (or strongly blocked) and State #5 strongly motivated, then the label would be an Instance of a self-centered, egotistic, pampered and self-indulgent position or individual. Another example may be an Instance where State #2 is strongly motivated and State #8 is weakly motivated, then the label would be that of an Instance of an extraverted, earnest position or individual yet highly introverted with a tendency to give up before any possibility is enabled.

Experiential Instances may preferably contain at least one reference to States #1-4 and at least one reference to States #5-8, as States #1-4 are typically more extravertive and deal with outer circumstances and evolving developments, while States #5-8 are typically more introvertive and deal with one's inner circumstance and challenges when facing such outer occurrences.

Once such set of reference labeled Instances are defined, combinations of them may further be used to model reference labeled Processes. Once Instance and Process references are defined and labeled, including their respective computed statistical parameters (e.g. Gaussian mean and standard deviation) over sufficient examples, the model may be applied to newly experienced Instances and their attribute values I_{T}(A). Said attribute values A are utilized to compute likelihoods of each attribute using the Gaussian distribution with respective modeled parameters, and the label with maximum probability is selected (i.e. maximum a posteriori or MAP decision rule).

The Naïve Bayes Classifier of experiential Instances is a conditional probability P( ) model, whereby the labeling of I_{T}(A), an experiential Instance at time T with attributes vector A, as a reference Instance IL is represented mathematically as follows:

P(I_{L}|I_{T}(A))αP(I_{L})ΠᵢP(Aᵢ|I_{L}) (Eq #3)

Where α stands for proportionality, i is the index of all features of I_{T}(A), and P(Aᵢ|I_{L}) are the computed probabilities of each of the Aᵢ features i for a Gaussian distribution of each label L with mean μ_{L,i} and standard deviation σ_{L,i},

P(Aᵢ|I_{L})=(½πσ_{L,i}²)^{1/2} exp(−(Aᵢ−μ_{L,i})²/2σ_{L,i}²) (Eq #4)

The labeled result I_{L} is selected based on the maximum posterior probability of all reference Instances P(I_{L})ΠᵢP(Aᵢ|I_{L}).

Another preferred embodiment of this invention is that of determining and localizing positive motivators (enablers, facilitators) and negative motivators (blockages, inhibitors) in any given experiential Instance, as well as same for enhanced or reduced flow and progressive evolution of an experiential Process comprising multiple accumulated experiential Instances.

Furthermore, the ratio

R=(Ai−μ_{L,i})/σ_{L,i} (Eq #5)

is useful in representing a distance measure from a given Label that reflects the facilitating (positive values) or inhibiting (negative values) intensities of the experiential features. For example, for the range of R values between −3 to +3, −3 would be considered an extreme inhibitor, −2 a medium inhibitor and −1 a moderate inhibitor, and similarly +3 would be considered a significant facilitator, +2 a medium facilitator and +1 a moderate facilitator. This can be particularly useful is providing indications and highlights to a user that reflect which experiential features are positive facilitators and which are negative inhibitors which require further attention in responding to specific experiential Instances and improving the flow of experiential Processes.

A preferred machine learning embodiment for Unit 2740 known in the art relies on a learning algorithm for a single-layer Perceptron. For multilayer Perceptrons, hidden layers are used in conjunction with more complex algorithms such as backpropagation. If the activation function or the underlying Instance or Process being modeled by the Perceptron is nonlinear, alternative learning algorithms may be used. Moreover, multiple Perceptrons may be combined in an artificial neural network.

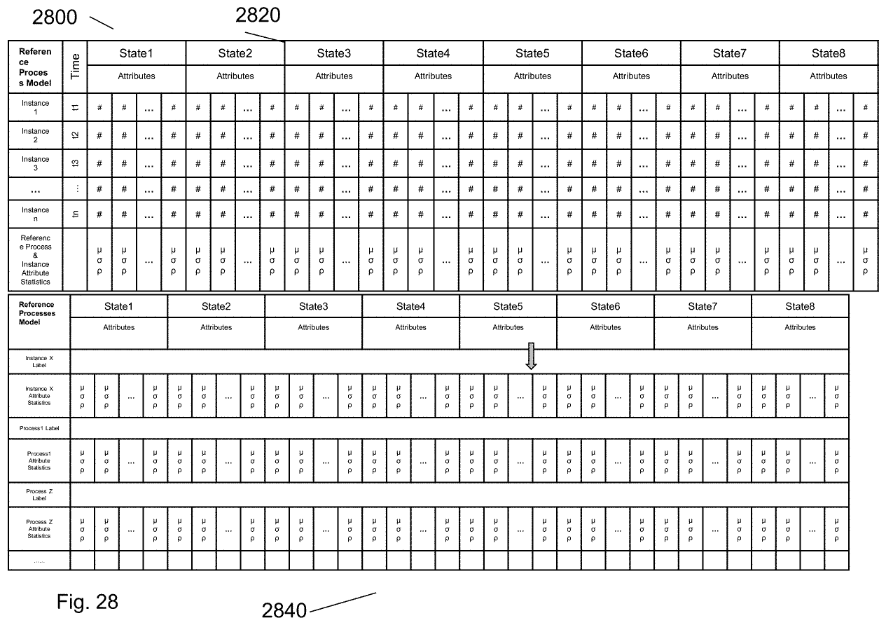

FIG. 28 Unit 2820 illustrates a structured data table of attribute entries of several Instances and a modeled Process, for each of the eight States and entered and accumulated in multiple Instances, thereby facilitating computing numerical statistical parameters (e.g. Mean μ, Std.Dev. σ, and Correlation/Regression ρ) for each of the said attributes in each State. Unit 1840 illustrates a structured data table of said computed statistics of multiple differently-labeled reference Processes and Instances that is useful in classifying and associating different input Instances to one of said labeled Processes or Instances, in accordance with this invention. The computed statistical parameters of experiential entries are useful in applying a Bayesian Naïve Classifier and associating such Instances to reference Instances and Processes.

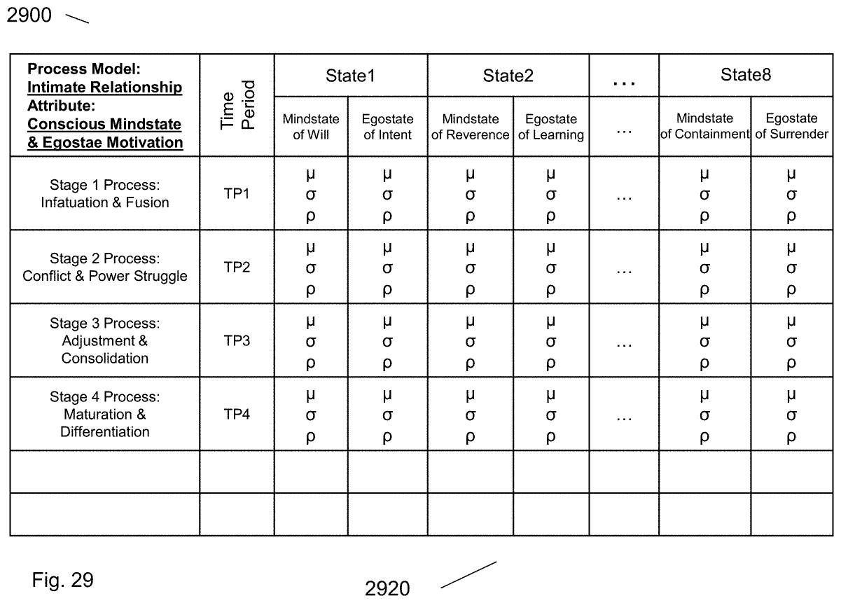

FIG. 29 Unit 2920 presents an exemplary structured data table of the attribute statistics of the conscious Mind and Ego mental motivators, compiled in time periods that reflect progressive designated stages of an intimate relationship Process model, for each of the eight States, thereby facilitating classification of Instances and Processes along with various assessments of said relationship Process, in accordance with this invention.

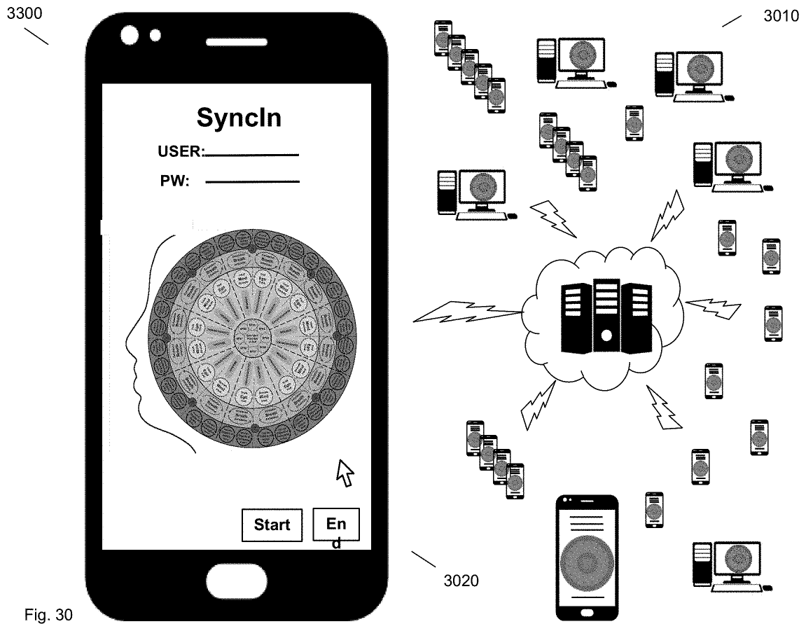

FIG. 30 illustrates a mobile cellular device application that facilitates the use of the experiential Construct in everyday life by an end user and by a professional therapist, and the app's connectivity to a central server and other devices, in accordance with this invention.

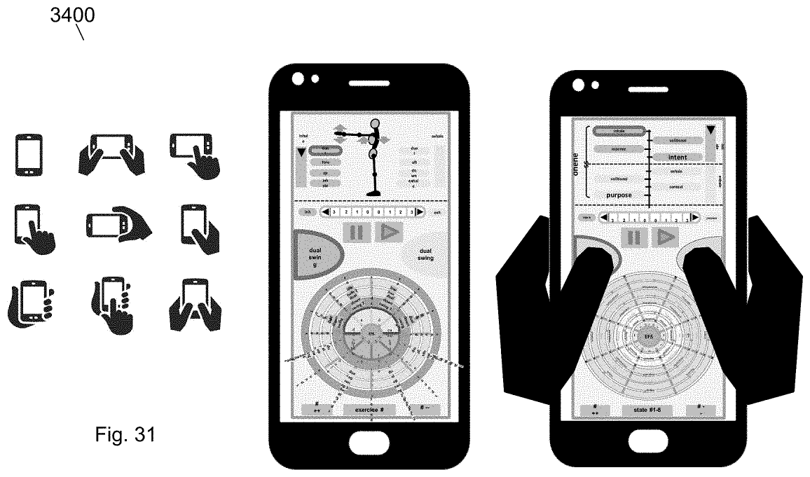

FIG. 31 illustrates an exemplary SyncIn APP display and user interfaces, in accordance with this invention.

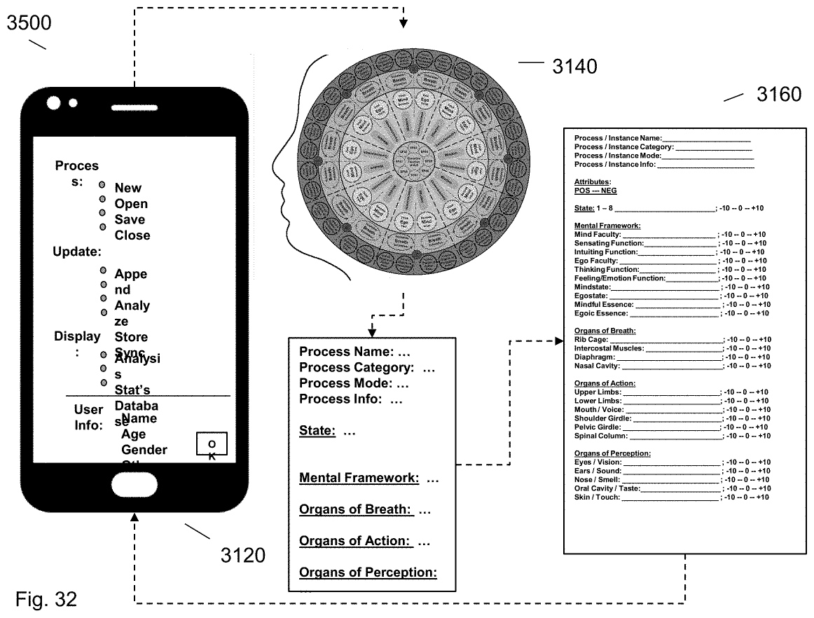

FIG. 32 illustrates the mobile device application as an experiential data entry assistant by using the graphical Construct, in accordance with this invention.

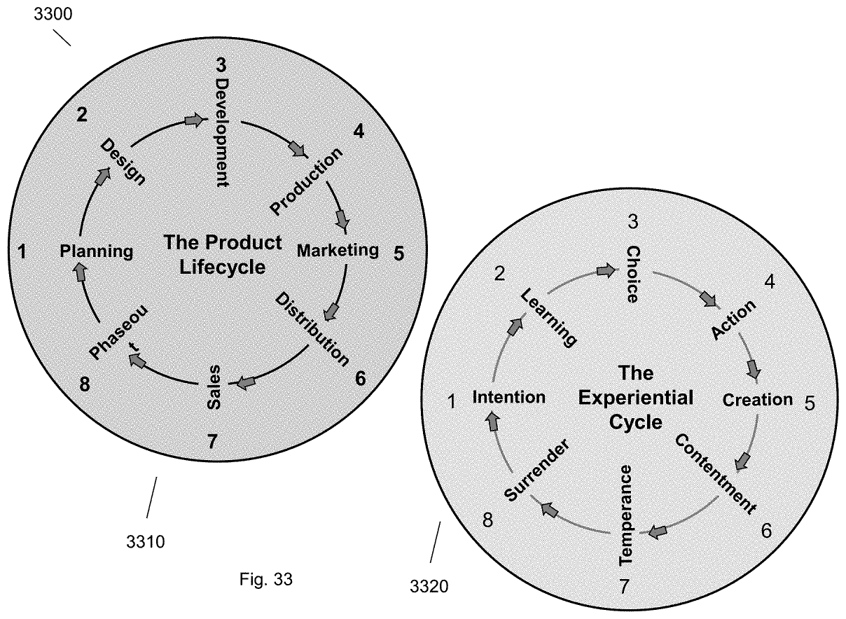

FIG. 33 illustrates another utility of the current invention, that associates the Experiential Process according to this invention (Unit 3320) with the Product Life Cycle (Unit 3310). By associating the EI's of both, improved employee identification with the workplace and respective product lifecycles on which they are working may be better determined. This also results in improved employee screening and placement at the workplace, hence increasing employees' mental wellbeing, work motivation, identification with products and projects in which they are involved, and general employment satisfaction and contribution.

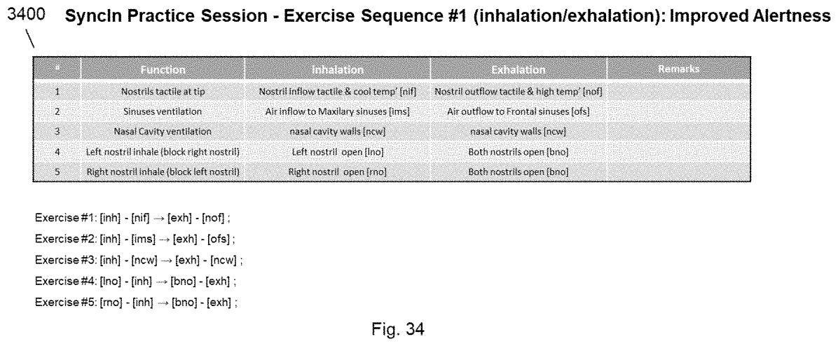

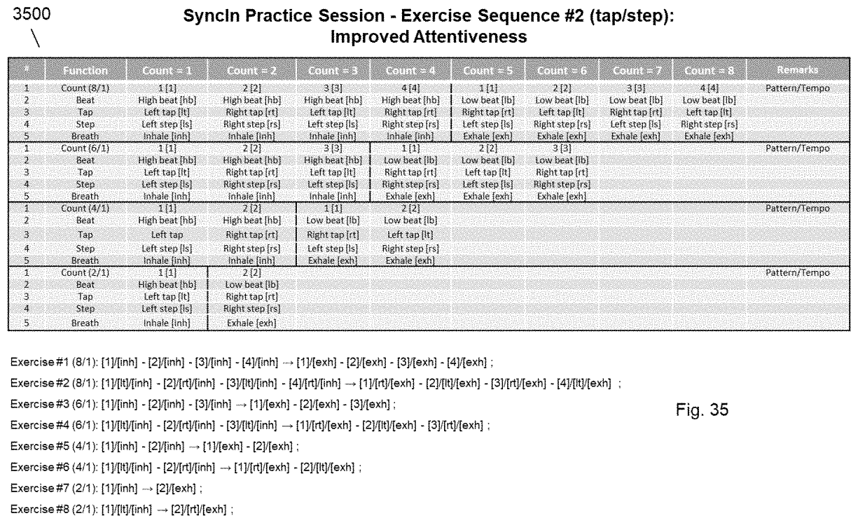

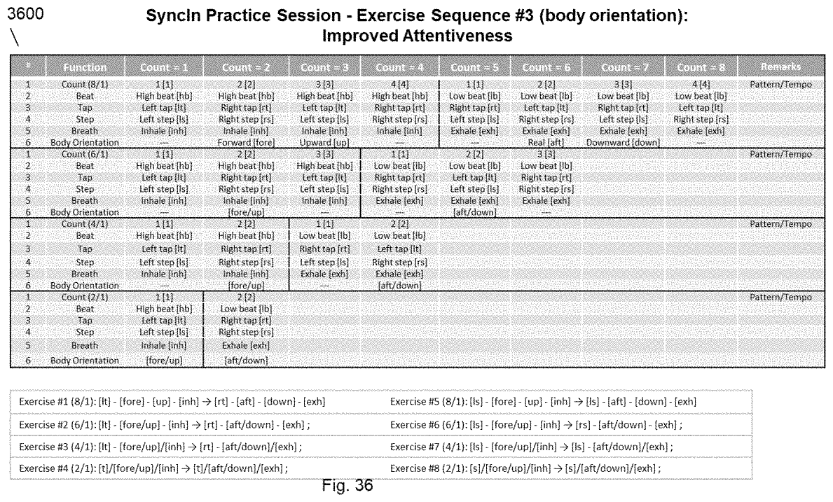

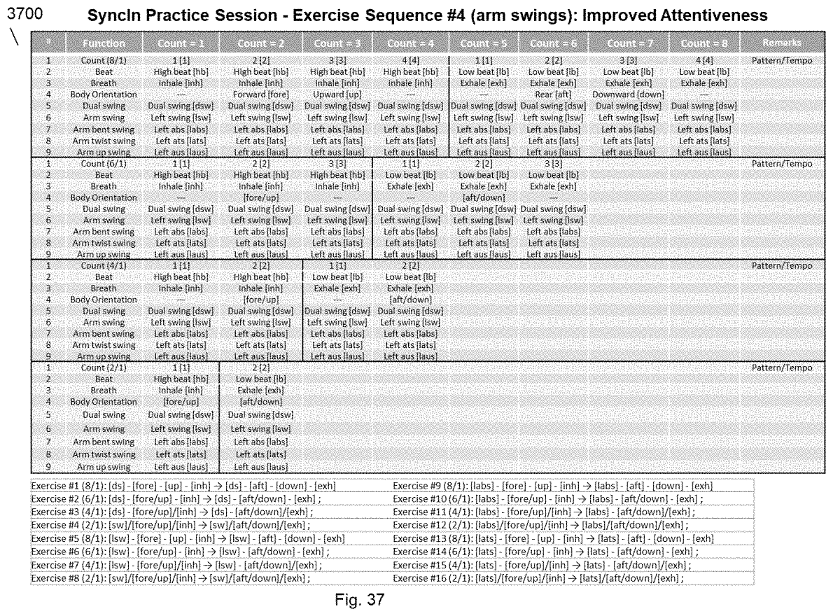

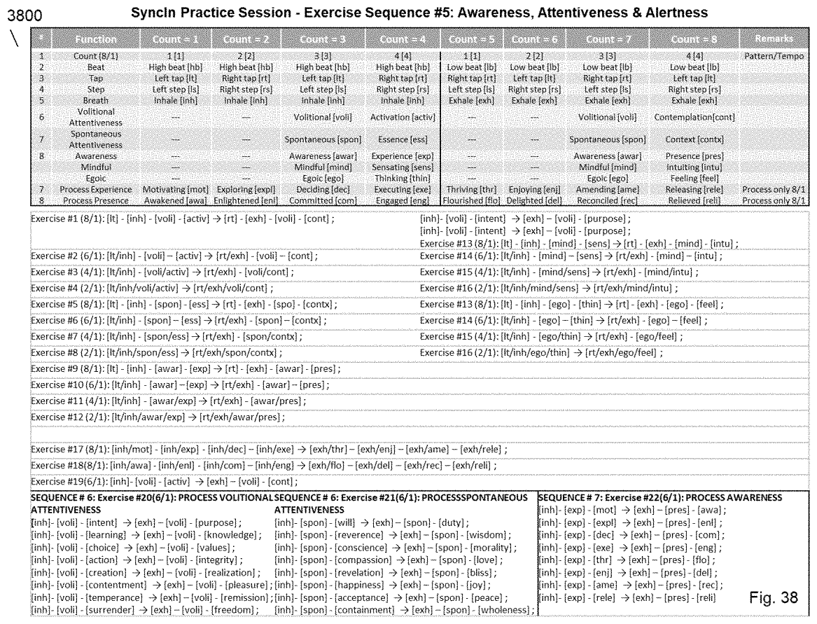

FIGS. 34-38 list for reference a variety of SyncIn practice sessions, including:
Exercise Sequence #1 (inhalation/exhalation): Improved Alertness
Exercise Sequence #2 (tap/step): Improved Attentiveness
Exercise Sequence #3 (body orientation): Improved Attentiveness
Exercise Sequence #4 (arm swings): Improved Attentiveness
Exercise Sequence #5: Awareness, Attentiveness & Alertness.

1-3. (canceled)

4. A system for improving synchronization of breath exercises, body exercises, and mind exercises of a user, the system comprising:
(a) a breath detecting module for tracking breath patterns of the user;
(b) a motion detecting module for tracking motion of the user;
(c) a speech detecting module for tracking speech patterns of the user, the speech patterns reflecting a mental exercise of the user;
(d) at least one output interface for providing output to the user;
(e) a timer; and
(f) a processor, functionally associated with the breath detecting module, the motion detecting module, the speech detecting module, the output interface, the timer, and the communication interface, the processor operative to:
i. select at least two exercises to be carried out synchronously by the user, the at least two exercises including exercises of at least two of the categories of breath exercises, body exercises, and mind exercises;
ii. determine a beat to which the at least two exercises are to be synchronized;
iii. guide the user, via the at least one output interface, to carry out an exercise sequence including the selected at least two exercises at the determined beat;
iv. receive input from at least two of the breath detecting module, the motion detecting module, and the speech detecting module, the input reflecting the user's carrying out the exercises;
v. process the received input, using the timer, to determine the degree of the user's accuracy or synchronization during carrying out of the selected exercises; and
vi. record in a user profile associated with the user a synchronization level achieved by the user during carrying out of the exercise sequence,
wherein, progressive user exercise using the system improves the synchronization of the user while carrying out selected exercise sequences.

5. The system according to claim 4, wherein the breath detecting module comprises an audio receiver.

6. The system according to claim 4, wherein the breath detecting module comprises an air-flow sensor.

7. The system according to claim 4, wherein the breath detecting module comprises an image capturing element adapted to capture images of the user, and wherein the processor is configured to identify, in the captured images, changes to the body of the user reflecting a breath pattern of the user.

8. The system according to claim 4, wherein the motion detecting module comprises an audio receiver adapted to track the motion when there is a sound generated by the motion.

9. The system according to claim 8, wherein the motion comprises the user tapping one or more fingers on a surface.

10. The system according to claim 4, wherein the motion detecting module comprises a tactile sensor adapted to sense the motion carried out by the user.

11. The system according to claim 10, wherein the tactile sensor comprises a touchpad or a touchscreen of a computing device.

12. The system according to claim 4, wherein the motion detecting module comprises an image capturing element adapted to capture images of the user, and wherein the processor is configured to identify, in the captured images, motions of the user.

13. The system according to claim 4, wherein the motion detecting module is adapted to detect the user tapping their fingers on a surface as the motion being tracked.

14. The system according to claim 4, wherein the motion detecting module is adapted to detect motion of the user's limbs as the motion being tracked.

15. The system according to claim 4, wherein the processor is configured to select the at least two exercises based on information stored in the user profile with respect to previous exercise sequences carried out by the user.

16. The system according to claim 4, wherein the processor is further configured to select a theme with respect to which the exercise sequence is carried out by the user.

17. The system according to claim 16, further comprising an input interface adapted to receive input from the user, and wherein the processor is further configured to:
present to the user, via the output interface, questions querying the user's mental and behavioral experiential intensities related to carrying out of the exercise sequence with respect to the theme;
in response to the questions, receive from the user, via the input interface, scores reflecting the user's mental and behavioral experiential intensities related to carrying out of the exercise sequence with respect to the theme; and
record in the user profile the user's perceived mental and behavioral experiential intensities.

18. The system according to claim 17, wherein the processor is configured to use machine learning tools to analyze the experiential intensities of the user to characterize and map the theme-related experiential intensities of the user, and to provide to the user, via the output interface, feedback with respect to their carrying out the exercise sequence or guidance for improving their carrying out of the exercise sequence, so as to increase the awareness of the user to such experiential intensities during carrying out of the exercise sequence.

19. The system according to claim 4, wherein the output interface includes a speaker adapted to provide to the user, during carrying out of the exercise sequence, beat sounds reflecting the determined beat of the exercise sequence and/or voice instructions.

20. The system according to claim 4, wherein the processor is configured to guide the user to carry out mental exercises by:
initially guiding the user to say exercise phrases, at the determined beat, using outer speech; and
progressively removing portions of the exercise phrases, to be completed by the user first using outer speech and later using inner speech,
wherein the combination inner and outer speech facilitates detection of the speech and thus of the synchronization of mental exercises carried out by the user with other selected exercises carried out by the user.

21. The system according to claim 20, wherein the exercises phrases reflect the type of exercise being carried out, wherein:
for movement exercises, the exercise phrase reflects the motion being carried out and the accompanying breath in accordance with the determined beat; and
for mental exercises, the exercise phrase reflects a meditative thought or experience being contemplated and a volitional attentiveness aspect of the user during carrying out of the exercise sequence.

22. The system according to claim 17, wherein the questions relate to theme-related, experiential intensity values of mental function dual pairs and emotion dual pairs, and the received scores relate to the experiential intensity values of the mental function dual pairs and emotion dual pairs, and wherein the processor is further configured to compute pairwise differential features for each of the dual pairs, and to map the computed features over the at least one theme or sequence of selected experiential themes or the at least one progression of states of a behavioral process.

23. The system according to claim 22, wherein:
the emotion dual pairs include at least one of:
anger-fear;
joy-sadness;
anticipation-surprise; and
trust-disgust; and
the mental function dual pairs that include at least one of:
thoughts-feelings;
sensations-intuitions;
thoughts-intuitions; and
sensations-feelings.

24. The system according to claim 23, wherein the processor is adapted to compute the experiential intensities of derived mental functions and emotions by RSS computation of respective dual pairs.

25. A method for improving synchronization of breath exercises, body exercises, and mind exercises of a user, the method comprising:
a) carrying out an initial user session, by:
i. selecting at least two exercises to be carried out synchronously by the user, the at least two exercises including exercises of at least two of the categories of breath exercises, body exercises, and mind exercises;
ii. determining a beat to which the at least two exercises are to be synchronized;
iii. guiding the user, via at least one output interface, to carry out an exercise sequence including the selected at least two exercises at the determined beat;
iv. receiving input from at least two of a breath detecting module, a motion detecting module, and a speech detecting module, the input reflecting the user's carrying out the exercises;
v. processing the received input, using a timer, to determine a degree of the user's accuracy or synchronization during carrying out of the selected exercises;
vi. recording, in a user profile associated with the user, a synchronization level achieved by the user during carrying out of the exercise sequence;
b) at a later time, carrying out a subsequent user session by repeating steps i to vi; and
c) comparing the synchronization level recorded in the initial user session with the synchronization level recorded in the subsequent user session to identify improvement in the synchronization of the user while carrying out selected exercise sequences.

26. A method for quantifying theme-based experiential cognitive, perceptive and/or emotional intensities of a user, the method comprising:
a) carrying out an initial user session, by:
i. receiving a selection of a desired beat, a breathing pattern, a theme, and an exercise sequence;
ii. guiding the user to perform inner speech synchronized dictations of cognitive, perceptive and/or emotional functions of the selected exercise sequence;
iii. receiving user inputs of the experiential cognitive, perceptive and/or emotional intensities;
iv. computing theme-based features of the experiential cognitive, perceptive and/or emotional intensities of the user based on the received user inputs;
v. creating a mapping and/or pattern representations of the computed theme-based features across designated theme sequences;
vi. recording, in a user profile associated with the user, the computed mapping and/or pattern representations;
b) at a later time, carrying out a subsequent user session by repeating steps i to vi; and
c) comparing the mapping and/or pattern representations recorded in the initial user session with the mapping and/or pattern representations recorded in the subsequent user session to identify variations of the user's theme-based experiential intensities while carrying out the selected exercise sequence.
